SEQUENCE LISTING (33 sequences)

SEQ ID NO: 1 - DNA, 2604 bp, Artificial Sequence
  Description of Artificial Sequence Synthetic polynucleotide
atgctgctatccgtgccgctgctgctcggcctcctcggcctggccgtcgccgagcctgccgtctacttcaaggagcagtttctggacggggacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctggaattcatgcaccagaagagaaccgccatgttccaggacccccaggagagacccaggaagctgccccagctgtgcaccgagctgcagaccaccatccacgacatcatcctggagtgcgtgtactgcaagcagcagctgctgaggagagaggtgtacgacttcgccttccgggacctgtgcatcgtgtacagagacggcaacccctacgccgtgggcgacaagtgcctgaagttctacagcaagatcagcgagtacagacactactgctacagcctgtacggcaccaccctggagcagcagtacaacaagcccctgtgcgacctgctgatccggtgcatcaacggccagaagcccctgtgccccgaggagaagcagcggcacctggacaagaagcagagattccacaacatcaggggccggtggaccggcagatgcatgagctgctgcaggagcagccggaccagacgggagacccagctgatgcacggcgacacccccaccctgcacgagtacatgctggacctgcagcccgagaccaccgacctgtacggctacggccagctgaacgacagcagcgaggaggaggacgagatcgacggccccgccggccaggccgagcccgaccgggcccactacaacatcgtgaccttctgctgcaagtgcgacagcaccctgaggctgtgcgtgcagagcacccacgtggacatcagaaccctggaggacctgctgatgggcaccctgggcatcgtgtgccccatctgcagccagaagccctctagaatgaagagggccagcgccacccagctgtacaagacctgcaagcaggccggcacctgcccccccgacatcatctccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacacc

SEQ ID NO: 2 - protein, 868 aa, Artificial Sequence
  Description of Artificial Sequence Synthetic peptide
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDELEFMHQKRTAMFQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINGQKPLCPEEKQRHLDKKQRFHNIRGRWTGRCMSCCRSSRTRRETQLMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPSRMKRASATQLYKTCKQAGTCPPDIISKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDT

SEQ ID NO: 3 - DNA, 297 bp, Human papillomavirus type 16
  CDS (1)..(297)
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaggataagctt

SEQ ID NO: 4 - protein, 99 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDKL

SEQ ID NO: 5 - protein, 98 aa, Human papillomavirus type 16
MHGDTPTLHEYMLDLQPETTDLYGYEGLNDSSEEEDEIDGPAGQAEPDRAHYNIVTFCCKCDSTLRLCVQSTHVDIRTLEDLLMGTLGIVCPICSQKP

SEQ ID NO: 6 - DNA, 477 bp, Human papillomavirus type 16
  CDS (1)..(474)
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 7 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 8 - protein, 151 aa, Human papillomavirus type 16
MFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 9 - DNA, 1422 bp, Human papillomavirus type 16
atgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctag

SEQ ID NO: 10 - protein, 473 aa, Human papillomavirus type 16
MRHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFITTPTKLITYDNPAYEGIDVDNTLYFSSNDNSINIAPDPDFLDIVALHRPALTSRRTGIRYSRIGNKQTLRTRSGKSIGAKVHYYYDFSTIDSAEEIELQTITPSTYTTTSHAALPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 11 - DNA, 1899 bp, Homo sapiens
gtccgtactgcagagccgctgccggagggtcgttttaaagggccgcgttgccgccccctcggcccgccatgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagcttgccgcgccaaataatgtctctgtgagactcgagaactttcatttttttccaggctggttcggatttggggtggattttggttttgttcccctcctccactctcccccaccccctccccgccctttttttttttttttttaaactggtattttatcctttgattctccttcagccctcacccctggttctcatctttcttgatcaacatcttttcttgcctctgtgccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctc

SEQ ID NO: 12 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 13 - protein, 26 aa, Human papillomavirus type 16
LSRHFMHQKRTAMFQDPQERPRKLPQ

SEQ ID NO: 14 - protein, 31 aa, Human papillomavirus type 16
AMFQDPQERPRKLPQLCTELQTTIHDIILEC

SEQ ID NO: 15 - protein, 22 aa, Human papillomavirus type 16
PTLHEYMLDLQPETTDLYCYEQ

SEQ ID NO: 16 - protein, 11 aa, Human papillomavirus type 16
HEYMLDLQPET

SEQ ID NO: 17 - protein, 15 aa, Human papillomavirus type 16
TLHEYMLDLQPETTD

SEQ ID NO: 18 - protein, 14 aa, Human papillomavirus type 16
EYMLDLQPETTDLY

SEQ ID NO: 19 - protein, 17 aa, Human papillomavirus type 16
DEIDGPAGQAEPDRAHY

SEQ ID NO: 20 - protein, 15 aa, Human papillomavirus type 16
GPAGQAEPDRAHYNI

SEQ ID NO: 21 - protein, 20 aa, Human papillomavirus type 16
TGVPIDPAVPDSSIVPLLES

SEQ ID NO: 22 - protein, 19 aa, Human papillomavirus type 16
GAEIEIAEVHPPPVYEGPE

SEQ ID NO: 23 - protein, 21 aa, Human papillomavirus type 16
VTIGDIEEPPILEVVPETHPT

SEQ ID NO: 24 - protein, 80 aa, Human papillomavirus type 16
SRMKRASATQLYKTCKQAGTCPPDIISKVEGKTIADQILQYGSMGVFFGGLGIGTGSGTGGRTGYIPLGTRPPTATDTLA

SEQ ID NO: 25 - protein, 88 aa, Human papillomavirus type 16
MKRASATQLYKTCKQAGTCPPDIISKVEGKTIADQILQYGSMGVFFGGLGIGTGSGTGGRTGYIPLGTRPPTATDTLAPVRPPLTVDP

SEQ ID NO: 26 - protein, 60 aa, Homo sapiens
MLLSVPLLLGLLGLAVAEPAVYFKEQFLDGDGWTSRWIESKHKSDFGKFVLSSGKFYGDE

SEQ ID NO: 27 - protein, 4 aa, Human papillomavirus type 16
QDKL

SEQ ID NO: 28 - DNA, 29 bp, Artificial Sequence
  Description of Artificial Sequence Synthetic primer
aaatctagaatgaagagggccagcgccaa

SEQ ID NO: 29 - DNA, 31 bp, Artificial Sequence
  Description of Artificial Sequence Synthetic primer
aaagcggccgctcaggtgtccatgggatctc

SEQ ID NO: 30 - DNA, 31 bp, Artificial Sequence
  Description of Artificial Sequence Synthetic primer
aaagtcgacatgctgctatccgtgccgctgc

SEQ ID NO: 31 - DNA, 26 bp, Artificial Sequence
  Description of Artificial Sequence Synthetic primer
gaattcgttgtctggccgcacaatca

SEQ ID NO: 32 - DNA, 28 bp, Artificial Sequence
  Description of Artificial Sequence Synthetic primer
tttgaattcatgaagagggccagcgcca

SEQ ID NO: 33 - DNA, 29 bp, Artificial Sequence
  Description of Artificial Sequence Synthetic primer
tttagatcttcaggtgtccatggggatct

(19) United States
(12) Patent Application Publication
(10) Pub. No.: US 2008/0260765 A1
(43) Pub. Date: Oct. 23, 2008

(54) HPV DNA Vaccines and Methods of Use Thereof

(75) Inventors: Tzyy-Choou Wu, Stevenson, MD (US); Chien-Fu Hung, Timonium, MD (US); Richard Roden, Severna Park, MD (US)

Correspondence Address: Foley Hoag, LLP (w/JHV), World Trade Center West, 155 Seaport Blvd, Boston, MA, 02210-2600, US

(73) Assignee: Johns Hopkins University, Baltimore, MD (US)

(21) Appl. No.: 12/049,596

(22) Filed: Mar. 17, 2008

Related U.S. Application Data
(60) Provisional application No. 60/918,278, filed on Mar. 15, 2007.

(51) Int. Cl. A61K 39/00; C07H 21/00; A61P 37/00

(52) U.S. Cl. 424/186.1; 536/23.72; 424/204.1

(57) ABSTRACT

Human papillomavirus (HPV) infection is the etiological factor for cervical cancer. Provided are HPV vaccines that generate a humoral immune response to prevent new infection, as well as cell-mediated immunotherapy to eliminate established infection or HPV-related disease. HPV vaccines include nucleic acid sequences encoding HPV16 early proteins E6 and E7. Additional nucleic acid sequences in the vaccines include sequences encoding calreticulin and/or the HPV16 late protein L2. Methods using these vaccines are provided that result in therapeutic effects.

### CROSS-REFERENCE TO RELATED APPLICATIONS

This application claims the benefit of U.S. Provisional Application No. 60/918,278, filed Mar. 15, 2007, the contents of which are specifically incorporated by reference herein.

### GOVERNMENT INTEREST

This invention was made using funds from grants from the U.S. National Institutes of Health, including grants CA098252 and CA114425-01. The U.S. government therefore retains certain rights in the invention.

### FIELD OF THE INVENTION

The present invention in the fields of molecular biology, immunology and medicine relates to nucleic acid vaccines for the prevention and treatment of diseases such as cancer. Specifically, treatment and prevention of cancers involving human papillomavirus or papilloma virus (HPV) infections are provided by vaccines that include nucleic acid sequences encoding HPV early proteins E6 and E7, the HPV late protein L2, and calreticulin.

### DESCRIPTION OF THE BACKGROUND ART

Cervical cancer is the 2^{nd} leading cause of cancer deaths in women worldwide. The primary factor in the development of cervical cancer is infection by the human papillomavirus. HPV is one of the most common sexually transmitted diseases in the world. It is now known that cervical cancer is a consequence of persistent infection with high-risk type HPV. HPV infection is a necessary factor for the development and maintenance of cervical cancer and thus, effective vaccination against HPV represents an opportunity to control cervical cancer.

Cytotoxic T lymphocytes (CTL) are critical effectors of anti-viral and anti-tumor responses (reviewed in Chen, C H et al., J Biomed Sci. 5: 231-252, 1998; Pardoll, D M. Nat Med. 4: 525-531, 1998; Wang, R F et al., Immunol Rev. 170: 85-100, 1999). Activated CTL are effector cells that mediate antitumor immunity by direct lysis of their target tumor cells or virus-infected cells and by releasing cytokines that orchestrate immune and inflammatory responses that interfere with tumor growth or metastasis, or viral spread. Depletion of CD8⁺ CTL leads to the loss of anti-tumor effects of several cancer vaccines (Lin, K-Y et al., Canc Res 56: 21-26, 1996; Chen, C-H et al., Canc Res. 60: 1035-42, 2000). Therefore, the enhancement of antigen presentation through the MHC class I pathway to CD8⁺ T cells has been a primary focus of cancer immunotherapy.

Naked DNA vaccines have emerged as attractive approaches for vaccine development (reviewed in Hoffman, S L et al., Ann N Y Acad Sci 772: 88-94, 1995; Robinson, H L. Vaccine 15: 785-787, 1997; Donnelly, J J et al., Annu Rev Immunol 15: 617-648, 1997; Klinman, D M et al., Immunity 11: 123-129, 1999; Restifo, N P et al., Gene Ther 7: 89-92, 2000; Gurunathan, S et al., Annu Rev Immunol 18: 927-974, 2000). DNA vaccines generate long-term cell-mediated immunity (reviewed in Gurunathan, S et al., Curr Opin Immunol 12: 442-447, 2000) and generate CD8⁺ T cell responses in vaccinated humans (Wang, R et al. Science 282: 476-480, 1998). However, one limitation of these vaccines is their lack of potency, since the DNA vaccine vectors generally do not have the intrinsic ability to be amplified and to spread in vivo, as do some replicating viral vaccine vectors. Furthermore, some tumor antigens such as the E7 and E6 proteins of human papillomavirus-16 (“HPV-16”) are viewed as weak immunogens. Therefore, there is a need in the art for strategies to enhance DNA vaccine potency, particularly for more effective cancer and viral immunotherapy.

Intradermal administration of DNA vaccines via gene gun can efficiently deliver genes of interest into professional antigen presenting cells (APCs) in vivo (Condon C et al., Nat Med, 2: 1122-28, 1996). The skin contains numerous bone marrow-derived APCs (called Langerhans cells) that are able to move through the lymphatic system from the site of injection to draining lymph nodes (LNs), where they can prime antigen-specific T cells (Porgador A et al., J Exp Med 188: 1075-1082, 1998). APCs exist in other sites, particularly in lymphatic tissue as dendritic cells (DC). Gene gun immunization therefore provides the opportunity to provide HPV vaccine strategies that involve direct delivery of nucleic acids (e.g., DNA or RNA) to APCs.

### SUMMARY OF THE INVENTION

This invention includes in one aspect a nucleic acid composition that contains a first nucleic acid including a first sequence encoding an E6 or E7 protein of a human papillomavirus (HPV), linked to a second sequence that encodes an HPV late protein L2, and a second nucleic acid encoding a calreticulin. The linkage between nucleic acid sequences can occur directly or via a linker.

In another aspect, the invention provides a nucleic acid composition that contains a first sequence encoding a human papillomavirus (HPV) E6 protein or an immunogenically active fragment thereof, a second sequence encoding a HPV E7 protein or an immunogenically active fragment thereof, a third sequence encoding an HPV late protein L2 or an immunogenically active fragment thereof, and a fourth sequence encoding a calreticulin or an immunogenically active fragment thereof. Preferably, the HPV is HPV-16. In certain embodiments, the calreticulin is human calreticulin. The nucleic acid composition may be contained in a plasmid vector, such as an expression vector, and an immunologically acceptable excipient or carrier. In certain embodiments the invention provides a nucleic acid composition bound to a particle suitable for introduction into a cell or an animal. For example, this particle is a gold particle.

The invention also provides a method of inducing or enhancing an antigen-specific immune response in a mammalian subject by administering to the subject an effective amount of a nucleic acid composition as described herein, thereby inducing or enhancing the antigen specific immune response.

The invention further provides a method of inducing or enhancing an antigen-specific immune response in a mammalian subject by administering to the subject an effective amount of a nucleic acid composition bound to a particle, thereby inducing or enhancing the antigen specific immune response. In certain embodiments, the antigen-specific immune response is mediated at least in part by CD8⁺ cytotoxic T lymphocytes (CTL). In other embodiments, the antigen specific immune response is mediated at least in part by CD8⁻ cytotoxic T lymphocytes. The mammalian subject is, for example, a human, such as a human having a tumor, and the nucleic acid composition or a particle containing the nucleic acid composition is administered intratumorally or peritumorally. The particle is capable of being administered intradermally by particle bombardment. The induced or enhanced immune response is preferably specific for HPV E6 or E7 protein or an immunogenically active fragment thereof. In other embodiments, the induced or enhanced immune response is, greater in magnitude than an immune response induced by a DNA that encodes HPV E6, E7 and L2 without a DNA encoding the calreticulin or fragment thereof.

The nucleic acid compositions of the present invention include nucleic acids encoding modified proteins, such as protein fragments. In certain embodiments, a HPV E6 protein contains the sequences LSRHFMHQKRTAMFQDPQERPRKLPQ or AMFQDPQERPRKLPQLCTELQTTIHDIILEC. In other embodiments, the HPV E7 protein contains the sequences PTLHEYMLDLQPETTDLYCYEQ, HEYMLDLQPET, TLHEYMLDLQPETTD, EYMLDLQPETTDLY, DEIDGPAGQAEPDRAHY or GPAGQAEPDRAHYNI. In still other embodiments, the HPV L2 protein includes the sequences TGVPIDPAVPDSSIVPLLES, GAEIEIAEVHPPPVYEGPE, VTIGDIEEPPILEVVPETHPT, SRMKRASATQLYKTCKQAGTCPPDIISKVEGKTIAD QILQYGSMGVFFGGLGIGTGSGTGGRTGYIPLGTRPPTATDTLA, or MKRASATQLYKTCKQAGTCPPDIISKVEGKTIADQILQYGSMGVFFGGLGIGTGSGTGGRT GYIPLGTRPPTATDTLAPVRPPLTVDP. In additional embodiments, the calreticulin protein contains the amino acid sequence MLLSVPLLLGLLGLAVAEPAVYFKEQFLDGDG WTSRWIESKHKSDFGKFVLSSGKFYGDE.

The present invention also provides a nucleic acid composition containing SEQ ID NO: 1, and a nucleic acid composition encoding an amino acid sequence that contains SEQ ID NO: 2.

### BRIEF DESCRIPTION OF THE DRAWINGS

FIG. 1 demonstrates the experimental characterization of expression of E6, E7 and L2 in DNA transfected cells. BHK 21 cells were transfected with 2 μg of the various DNA constructs, pNGVL4a, pNGVL4a-L2, pNGVL4a-hCRT, pNGVL4a-hCRTL2, pNGVL4a-hCRTE6E7 or pNGVL4a-hCRTE6E7L2 as described herein. FIG. 1 shows a photograph of Western blot analysis performed using 50 μg of protein from the cell lysates 24 hours after transfection. Protein expression was determined using mouse anti-HPV 16 E6 (upper panel), E7 (middle panel) or rabbit anti-HPV L2 (lower panel) polyclonal antibody. Western blot analysis showing the expression of HPV 16 E6, E7 and L2 proteins in BHK 21 cells transfected with each of the six DNA constructs. The arrows indicate the molecular weights. Expression of E6 and E7 proteins was observed in the 4a-hCRTE6E7 (lane 5) and the 4a-hCRTE6E7L2 (lane 6) constructs. Expression of L2 protein was observed in the 4a-L2 (lane 2), 4a-hCRTL2 (lane 4) and the 4a-hCRTE6E7L2 (lane 6) constructs.

FIG. 2 demonstrates the characterization of IFNγ-secreting E6-specific CD8+ T cell precursors in mice vaccinated with recombinant DNA vaccines. One set of C57BL/6 mice (3 per group) was immunized intradermally via gene gun with 2 μg of DNA per mouse of the various recombinant DNA vaccines, four times with one-week intervals. Another set of C57BL/6 mice (3 per group) was immunized intramuscularly with 50 μg per mouse of the various recombinant DNA vaccines four times with one-week intervals. The splenocytes from vaccinated mice were incubated with H-2K^{b}-restricted E6 CTL peptide (aa 50-57) overnight. Determination of the E6-specific CD8+ T cells was performed by intracellular IFN-γ staining followed by flow cytometry analysis. FIG. 2A shows a series of graphs of representative flow cytometry data showing the number of E6-specific IFNγ+ CD8+ T cells in the mice vaccinated either intradermally via gene gun (left panel) or intramuscularly (right panel) with the various DNA vaccines. FIG. 2B provides a bar graph showing the number of E6-specific IFNγ+ CD8+ T cells from mice immunized intradermally with the various DNA vaccines via gene gun with (shaded bars) or without (empty bars) stimulation with the E6 peptide. The data are shown as mean ±s.d. (p<0.05). FIG. 2C provides a bar graph showing the number of E6-specific IFNγ+ CD8+ T cells from mice immunized intramuscularly with the various DNA vaccines with (shaded bars) or without (empty bars) stimulation with the E6 peptide. The data are shown as mean ±s.d. (p<0.05).

FIG. 3 demonstrates the characterization of IFNγ-secreting E7-specific CD8+ T cell precursors in mice vaccinated with recombinant DNA vaccines. One set of C57BL/6 mice (3 per group) was immunized intradermally via gene gun with 2 μg of the various recombinant DNA vaccines four times with one-week intervals. Another set of C57BL/6 mice (3 per group) was immunized intramuscularly with 50 μg of the various recombinant DNA vaccines four times with one-week intervals. The splenocytes from vaccinated mice were incubated with H-2D^{b}-restricted E7 CTL peptide (aa 49-57) overnight. Determination of the E7-specific CD8+ T cells was performed by intracellular IFN-γ staining followed by flow cytometry analysis. FIG. 3A is a series of images providing representative flow cytometry data showing the number of E7-specific IFNγ+ CD8+ T cells in the mice vaccinated either intradermally via gene gun (left panel) or intramuscularly (right panel) with the various DNA vaccines. FIG. 3B is a bar graph showing the number of E7-specific IFNγ+ CD8+ T cells from mice immunized intradermally with the various DNA vaccines via gene gun with (shaded bars) or without (empty bars) stimulation with the E7 peptide. The data are shown as mean ±s.d. (p<0.05). FIG. 3C is a bar graph showing the number of E7-specific IFNγ+ CD8+ T cells from mice immunized intramuscularly with the various DNA vaccines with (shaded bars) or without (empty bars) stimulation with the E7 peptide. The data are shown as mean ±s.d. (p<0.05).

FIG. 4 demonstrates in vivo tumor protection and treatment. FIG. 4A is a graph showing an in vivo tumor protection experiment as described herein. C57BL/6 mice (5 per group) were immunized intradermally with the various DNA vaccines via gene gun twice with a one-week interval. Seven days after the last immunization, the mice were challenged subcutaneously with 5×10⁴ cells/mouse of TC-1 tumor cells. Tumor growth was monitored by visual inspection and palpation twice a week and examined by Kaplan-Meier analysis. FIG. 4B is a bar graph depicting the quantification of the number of pulmonary nodules in mice treated with the various DNA vaccines. C57BL/6 mice (5 per group) were challenged with 1×10⁴/mouse of TC-1 cells via the tail vein. Three and ten days later, the mice were treated intradermally with the various recombinant DNA vaccines via gene gun. Mice were sacrificed 28 days after tumor challenge and the numbers of pulmonary tumor nodules were quantified and compared. The data are shown as mean ±SE. (p<0.01).

FIG. 5 provides the characterization of antibody levels against full length L2 protein by ELISA. One group of C57BL/6 mice (3 per group) was immunized intradermally via gene gun with 2 μg of the various recombinant DNA vaccines four times with one-week intervals. Another group of C57BL/6 mice (3 per group) was immunized intramuscularly with 50 μg of the various recombinant DNA vaccines four times with one-week intervals. ELISA analysis was performed to determine the L2-specific antibody responses in vaccinated mice. FIG. 5A is a bar graph depicting the L2-specific antibody responses in mice vaccinated intradermally with the various DNA vaccines via gene gun. FIG. 5B is a bar graph depicting the L2-specific antibody responses in mice vaccinated intramuscularly with the various DNA vaccines. The results from 1:100, 1:500 and 1:1000 dilutions are represented, showing mean absorbance (OD 450 nm)±s.d. The data collected from all of the above experiments are from one representative experiment of two performed. Serum from a rabbit vaccinated with L2 was used as standard control.

FIG. 6 shows the results of neutralization assays using HPV-16 pseudovirions as demonstrated by graphical representations of the neutralization activity in mice vaccinated with the various DNA vaccines. FIG. 6A shows vaccination using gene gun delivery. One set of C57BL/6 mice (3 per group) was immunized intradermally via gene gun with 2 μg of the various recombinant DNA vaccines four times with one-week intervals. FIG. 6B shows vaccination using intramuscular injection. Another set of C57BL/6 mice (3 per group) was immunized intramuscularly with 50 μg of the various recombinant DNA vaccines four times with one-week intervals. Neutralizing assays were performed using HPV-16 pseudovirion to determine the L2-specific neutralizing antibody responses in vaccinated mice. RG1 is the 17-36aa anti-peptide serum that is used as a positive control antibody that generates a neutralizing response. B6I is the non-neutralizing negative control antibody.

### DETAILED DESCRIPTION

Cervical cancer is one of the most common cancers in women worldwide. Persistent infection with human papillomavirus (HPV) is considered to be the etiological factor for cervical cancer. Provided herein are effective vaccines against HPV infections that lead to the control of cervical cancer. Beneficially, HPV vaccines generate both humoral immune response to prevent new infections as well as cell-mediated immunity to eliminate established infection or HPV-related disease. Disclosed herein are preventive and therapeutic HPV DNA vaccines using human calreticulin (CRT), HPV16 early proteins E6 and E7, and the HPV late protein L2. The DNA vaccines described herein generate effective CTL and antibody responses by delivering antigens to APCs that stimulate CD4+ and CD8+ T cells. Compared to live viral or bacterial vectors, naked DNA plasmid vaccines are safe and can be easily administered. Furthermore, DNA vaccines are easy to prepare on a large scale with high purity and high stability and can be engineered to express antigenic peptides or proteins. Additionally, DNA has the unique ability to be maintained long term in an episomal form. This enables prolonged expression of antigens and enhancement of immunologic memory. Using DNA vaccines to express proteins bypasses MHC restriction and thus maintains higher CTL responses than current protein vaccines. DNA vaccines can also be repeatedly applied to the same patient safely and effectively, unlike live vector vaccines. These features make DNA vaccines a useful approach for HPV vaccine development. While DNA has considerable advantages, one major limitation is its limited potency. This is due to the fact that DNA vaccines, unlike viral vectors, lack the intrinsic ability to amplify in transfected cells.

Calreticulin (CRT) is an abundant 46 kDa Ca²⁺-binding protein which is located in the endoplasmic reticulum (ER). CRT is considered to be related to the family of heat shock proteins (HSPs). This protein has been shown to associate with peptides delivered into the ER by transporters associated with antigen processing (TAP-1 and TAP-2) and with MHC class I-β2 microglobulin molecules to aid in antigen presentation. CRT has been previously employed to create effective DNA vaccines using HPV-16 E6, E7 or SARS-Co-V as target antigens. The inventors have shown that DNA vaccines encoding calreticulin (CRT) linked to the target antigen generate the high levels of antigen-specific CD8⁺ T-cell responses as well as significant antigen-specific humoral immunity. Thus, CRT is used in a HPV DNA vaccine development in order to generate strong T cell specific responses as well as humoral responses against the HPV antigens, E6, E7 and L2. The employment of E6, E7 and L2 protein of HPV16 has been previously explored in protein-based vaccines to generate a chimeric L2E7E6 fusion protein (also called TA-CIN). Vaccination of healthy volunteers with TA-CIN induced serum antibody that neutralizes across papillomavirus species.

In the current invention, an HPV DNA vaccine is provided that encodes calreticulin linked to HPV16 early proteins, E6 and E7, and the late protein L2 (hCRTE6E7L2). It is demonstrated that vaccination with hCRTE6E7L2 DNA vaccine induces a potent E6/E7-specific CD8+ T cell immune response and results in a significant therapeutic effect against E6/E7-expressing tumor cells. Furthermore, vaccination with hCRTE6E7L2 generates significant L2-specific neutralizing antibody responses against HPV-16 pseudovirion infection. Thus, the hCRTE6E7L2 DNA vaccines generate potent preventive and therapeutic effects in vaccinated mice. However, the present invention is not limited to the exemplified antigens. Rather, one of skill in the art will appreciate that the same results are expected for any antigen (and epitopes thereof) for which a T cell-mediated response is desired. The response so generated will be effective in providing protective or therapeutic immunity, or both, directed to an organism or disease in which the epitope or antigenic determinant is involved—for example as a cell surface antigen of a pathogenic cell or an envelope or other antigen of a pathogenic virus, or a bacterial antigen, or an antigen expressed as or as part of a pathogenic molecule.

Unless defined otherwise, all technical and scientific terms used herein have the meaning commonly understood by a person skilled in the art of this invention. As used herein, the following terms have the meanings ascribed to them unless specified otherwise.

The term “antigen” or “immunogen” as used herein refers to a compound or composition or cell comprising a peptide, polypeptide or protein which is “antigenic” or “immunogenic” when administered in an appropriate amount (an “immunogenically effective amount”), i.e., capable of inducing, eliciting, augmenting or boosting a cellular and/or humoral immune response and of being recognized by the products of that response (T cells, antibodies). A nucleic acid such as DNA that encodes an immunogen and is used as a vaccine is referred to as a “DNA immunogen,” as the encoded polypeptide is expressed in vivo after administration of the DNA. An immunogen may be effective when given alone or in combination, or linked to, or fused to, another substance (which can be administered at one time or over several intervals). An immunogenic composition can comprise an antigenic peptide/polypeptide of at least about 5, or about 10 or about 15, or about 20 amino acids, etc. Smaller antigens may require presence of a “carrier” polypeptide e.g., as a fusion protein, aggregate, conjugate or mixture, preferably linked (chemically or otherwise) to the antigen to be immunogenic. The immunogen can be recombinantly expressed from a vaccine vector, which can be naked DNA which comprises the polypeptide immunogen's coding sequence operably linked to a promoter, e.g., an expression vector or cassette as described herein. The immunogen includes one or more antigenic determinants or epitopes which may vary in size from about 3 to about 15 or more amino acids.

The term “epitope” as used herein refers to an antigenic determinant or antigenic site that interacts with an antibody or a T cell receptor (TCR), e.g., MHC class I-binding peptides used in the methods of the invention. The specific conformational or stereochemical “domain” to which an antibody or a TCR bind is an “antigenic determinant” or “epitope.” TCRs bind to peptide epitopes which are physically associated with a third molecule, a major histocompatibility complex (MHC) class I or class II protein.

The term “recombinant” refers to (1) a nucleic acid or polynucleotide synthesized or otherwise manipulated in vitro or ex vivo, (2) methods of using recombinant DNA technology to produce gene products in cells or other biological systems, or (3) a polypeptide encoded by a recombinant nucleic acid. For example, the hCRTE6E7L2-encoding nucleic acid or polypeptide can be recombinant. “Recombinant means” includes ligation of nucleic acids having various coding regions or domains or promoter sequences from different sources into a single unit in the form of an expression cassette or vector for expression of the coding sequences in the vectors resulting in production of the encoded polypeptide. In one embodiment, the isolated or recombinant nucleic acid molecule is operatively linked to a promoter, such as, e.g., a constitutive, an inducible or a tissue-specific promoter. The promoter can be expressed in any cell, including cells of the immune system, including, e.g., antigen presenting cells (APCs), e.g., in a constitutive, an inducible or a tissue-specific manner. In alternative embodiments, the APCs are DCs, keratinocytes, astrocytes, monocytes, macrophages, B lymphocytes, a microglial cell, or activated endothelial cells, and the like. A “linker” is a moiety such as a nucleic acid or amino acid sequence that operably connects a first nucleic acid sequence with a second nucleic acid sequence, or connects a first amino acid sequence with a second amino acid sequence. Linkers may be cleavable by endonucleases or peptidases. Linkers may contain additional functionality, such as a label or attachment site. A nucleic acid linker may be from 1 to about 120 nucleotides in length; an amino acid linker may be from 1 to about 60 amino acid residues in length.

### Vectors, Antigen, and IPP Nucleic Acids and Polypeptides

### Plasmid Sequences

Plasmids used herein include the pET28a and pNGVL4a vectors as described in Example 1. Any nucleic acid expression vector can be employed in the present invention. pNGVL4a, a preferred plasmid backbone for the present invention was originally derived from the pNGVL3 vector, which has been approved for human vaccine trials. The pNGVL4a vector includes two immunostimulatory sequences (tandem repeats of CpG dinucleotides) in the noncoding region. Whereas any other plasmid DNA that can transform either APCs, preferably DC's or other cells which, via cross-priming, transfer the antigenic moiety to DCs, is useful in the present invention, pNGFVLA4a is preferred because of the fact that it has already been approved for human therapeutic use.

### Antigen Polypeptide Sequences

The present invention includes a combined DNA vaccine composition that includes multiple DNA sequences encoding antigenic polypeptides, which are termed “DNA immunogens” herein. For example, a DNA vaccine includes a DNA sequence encoding an E6 or an E7 immunogen, a DNA sequence encoding L2, and a DNA sequence encoding a calreticulin. In another example, the DNA vaccine encodes calreticulin, E6, E7, and L2. In any DNA vaccine containing multiple DNA sequences, the relative order of the DNA sequences (and thus the order of the expressed polypeptides) can be altered by one of skill in the art so long as the immunogenicity of each polypeptide is not eliminated.

### DNA Encoding HPV E7

The E7 nucleic acid sequence and amino acid sequence from HPV-16 are shown below (see Accession Number NC_{—}001526) (SEQ ID NO: 3)

| | |
|---|---|
| atg cat gga gat aca cct aca ttg cat gaa tat atg tta gat ttg caa cca gag aca act | 60 |
| Met His Gly Asp Thr Pro Thr Leu His Glu Tyr Met Leu Asp Leu Gln Pro Glu Thr Thr | 20 |
| gat ctc tac tgt tat gag caa tta aat gac agc tca gag gag gag gat gaa ata gat ggt | 120 |
| Asp Leu Tyr Cys Tyr Glu Gln Leu Asn Asp Ser Ser Glu Glu Glu Asp Glu Ile Asp Gly | 40 |
| cca gct gga caa gca gaa ccg gac aga gcc cat tac aat att gta acc ttt tgt tgc aag | 180 |
| Pro Ala Gly Gln Ala Glu Pro Asp Arg Ala His Tyr Asn Ile Val Thr Phe Cys Cys Lys | 60 |
| tgt gac tct acg ctt cgg ttg tgc gta caa agc aca cac gta gac att cgt act ttg gaa | 240 |
| Cys Asp Ser Thr Leu Arg Leu Cys Val Gln Ser Thr His Val Asp Ile Arg Thr Leu Glu | 80 |
| gac ctg tta atg ggc aca cta gga att gtg tgc ccc atc tgt tct cag gat aag ctt | 297 |
| Asp Leu Leu Met Gly Thr Leu Gly Ile Val Cys Pro Ile Cys Ser Gln Asp Lys Leu | 99 |

In single letter code, the wild type E7 amino acid sequence is

(SEQ ID NO:4)

| | |
|---|---|
| MHGDTPTLHE YMLDLQPETT DLYCYEQLND SSEEEDEIDG | 99 |
| PAGQAEPDRA HYNIVTFCCK CDSTLRLCVQ STHVDIRTLE | |
| DLLMGTLGIV CPICSQDKL | |

In another embodiment (See GenBank Accession No. AF125673, nucleotides 562-858 and the E7 amino acid sequence) the C-terminal four amino acids QDKL (and their codons) above are replaced with the three amino acids QKP (and the codons cag aaa cca) yielding a protein of 98 residues.

When an oncoprotein or an epitope thereof is the immunizing moiety, it is preferable to reduce the tumorigenic risk of the vaccine itself. Because of the potential oncogenicity of the HPV E7 protein, the E7 protein is preferably used in a “detoxified” form.

For example, to reduce oncogenic potential of E7 in a construct of this invention, one or more of the following positions of E7 is mutated:

| | | Preferred | nt Position | |
|---|---|---|---|---|
| Original | Mutant | codon | (in SEQ ID | Amino acid (in |
| residue | residue | mutation | NO: 3) | SEQ ID NO: 4) |
| Cys | Gly (or Ala) | TGT→GGT | 70 | 24 |
| Glu | Gly (or Ala) | GAG→GGG | 77 | 26 |
| | | (or GCG) | | |
| Cys | Gly (or Ala) | TGC→GGC | 271 | 91 |

The preferred E7 (detox) mutant sequence has the following two mutations: a TGT→GGT mutation resulting in a Cys→Gly substitution at position 24 of SEQ ID NO:4 a and GAG→GGG mutation resulting in a Glu→Gly substitution at position 26 of SEQ ID NO:4. This mutated amino acid sequence is shown below with the replacement residues underscored.

(SEQ ID NO:5)

| | |
|---|---|
| MHGDTPTLHE YMLDLQPETT DLYGYEGLND SSEEEDEIDG | 97 |
| PAGQAEPDRA HYNIVTFCCK CDSTLRLCVQ STHVDIRTLE | |
| DLLMGTLGIV CPICSQKP | |

These substitutions completely eliminate the capacity of the E7 to binding capacity to Rb, and thereby nullify its transforming activity.

Any nucleotide sequence that encodes encoding the above E7 or E7 (detox) polypeptide, or an antigenic fragment or epitope thereof, can be used in the present compositions and methods.

### DNA Encoding HPV E6

The wild type E6 nucleotide (SEQ ID NO:6) and amino acid (SEQ ID NO:7) sequences are shown below (see GenBank accession #'s K02718 and NC_{—}001526)):

| | |
|---|---|
| atg cac caa aag aga act gca atg ttt cag gac cca cag gag cga ccc aga aag tta cca | 60 |
| Met His Gln Lys Arg Thr Ala Met Phe Gln Asp Pro Gln Glu Arg Pro Arg Lys Leu Pro | 20 |
| cag tta tgc aca gag ctg caa aca act ata cat gat ata ata tta gaa tgt gtg tac tgc | 120 |
| Gln Leu Cys Thr Glu Leu Gln Thr Thr Ile His Asp Ile Ile Leu Glu Cys Val Tyr Cys | 40 |
| aag caa cag tta ctg cga cgt gag gta tat gac ttt gct ttt cgg gat tta tgc ata gta | 180 |
| Lys Gln Gln Leu Leu Arg Arg Glu Val Tyr Asp Phe Ala Phe Arg Asp Leu Cys Ile Val | 60 |
| tat aga gat ggg aat cca tat gct gta tgt gat aaa tgt tta aag ttt tat tct aaa att | 240 |
| Tyr Arg Asp Gly Asn Pro Tyr Ala Val Cys Asp Lys Cys Leu Lys Phe Tyr Ser Lys Ile | 80 |
| agt gag tat aga cat tat tgt tat agt ttg tat gga aca aca tta gaa cag caa tac aac | 300 |
| Ser Glu Tyr Arg His Tyr Cys Tyr Ser Leu Tyr Gly Thr Thr Leu Glu Gln Gln Tyr Asn | 100 |
| aaa ccg ttg tgt gat ttg tta att agg tgt att aac tgt caa aag cca ctg tgt cct gaa | 360 |
| Lys Pro Leu Cys Asp Leu Leu Ile Arg Cys Ile Asn Cys Gln Lys Pro Leu Cys Pro Glu | 120 |
| gaa aag caa aga cat ctg gac aaa aag caa aga ttc cat aat ata agg ggt cgg tgg acc | 420 |
| Glu Lys Gln Arg His Leu Asp Lys Lys Gln Arg Phe His Asn Ile Arg Gly Arg Trp Thr | 140 |
| ggt cga tgt atg tct tgt tgc aga tca tca aga aca cgt aga gaa acc cag ctg taa | 474 |
| Gly Arg Cys Met Ser Cys Cys Arg Ser Ser Arg Thr Arg Arg Glu Thr Gln Leu stop | 158 |

The wild type HPV E6 amino acid sequence (see GenBank Accession Number NC_{—}001526) (SEQ ID NO:7) is shown below. This sequence has 158 amino acids.

| | |
|---|---|
| MHQKRTAMFQ DPQERPRKLP QLCTELQTTI HDIILECVYC | 158 |
| KQQLLRREVY DFAFRDLCIV YRDGNPYAVC DKCLKFYSKI | |
| SEYRHYCYSL YGTTLEQQYN KPLCDLLIRC INCQKPLCPE | |
| EKQRHLDKKQ RFHNIRGRWT GRCMSCCRSS RTRRETQL | |

E6 proteins from cervical cancer-associated HPV types such as HPV-16 induce proteolysis of the p53 tumor suppressor protein through interaction with E6-AP. Human mammary epithelial cells (MECs) immortalized by E6 display low levels of p53. HPV-16 E6 as well as other cancer-related papillomavirus E6 proteins also binds the cellular protein E6BP (ERC-55). As with E7, it is preferred to used a non-oncogenic mutated form of E6, referred to as “E6 (detox).” Several different E6 mutations and publications describing them are discussed below.

The preferred amino acid residues to be mutated are underscored in the E6 amino acid sequence above. Some studies of E6 mutants are based upon a shorter E6 protein of 151 nucleic acids, wherein the N-terminal residue was considered to be the Met at position 8 in SEQ ID NO: 7 above. That shorter version of E6 is shown below as SEQ ID NO:8.

MFQDPQERPR KLPQLCTELQ TTIHDIILEC VYCKQQLLRR
EVYDFAFRDL CIVYRDGNPY AVCDKCLKFY SKISEYRHYC
YSLYGTTLEQ QYNKPLCDLL IRCINCQKPL CPEEKQRHLD
KKQRFHNIRG RWTGRCMSCC RSSRTRRETQ L

To reduce oncogenic potential of E6 in a construct of this invention, one or more of the following positions of E6 is mutated:

| Original | Mutant | aa position in | aa position in |
|---|---|---|---|
| residue | residue | SEQ ID NO: 7 | SEQ ID NO: 8 |
| Cys | Gly (or Ala) | 70 | 63 |
| Cys | Gly (or Ala) | 113 | 106 |
| Ile | Thr | 135 | 128 |

Nguyen M et al., J Virol. 6:13039-48, 2002, described a mutant of HPV-16 E6 deficient in binding α-helix partners, which displays reduced oncogenic potential in vivo. This mutant, that involves a replacement of Ile with Thr as position 128 (of SEQ ID NO:8), may be used in accordance with the present invention to make an E6 DNA vaccine that has a lower risk of being oncogenic. This E6(I¹²⁸T) mutant is defective in its ability to bind at least a subset of α-helix partners, including E6AP, the ubiquitin ligase that mediates E6-dependent degradation of the p53 protein.

Cassetti M C et al., Vaccine 22:520-52, 2004, examined the effects of mutations of four or five amino acid positions in E6 and E7 to inactivate their oncogenic potential. The following mutations were examined: E6-C⁶³G and E6 C¹⁰⁶ G (positions based on SEQ ID NO:8); E7-C²⁴ G, E7-E²⁶G, and E7 C⁹¹G (positions based on SEQ ID NO:4). Venezuelan equine encephalitis virus replicon particle (VRP) vaccines encoding mutant or wild type E6 and E7 proteins elicited comparable CTL responses and generated comparable antitumor responses in several HPV16 E6(+)E7(+) tumor challenge models: protection from either C3 or TC-1 tumor challenge was observed in 100% of vaccinated mice. Eradication of C3 tumors was observed in approximately 90% of the mice. The predicted inactivation of E6 and E7 oncogenic potential was confirmed by demonstrating normal levels of both p53 and Rb proteins in human mammary epithelial cells infected with VRPs expressing mutant E6 and E7 genes.

The HPV16 E6 protein contains two zinc fingers important for structure and function; one cysteine (C) amino acid position in each pair of C-X-X-C (where X is any amino acid) zinc finger motifs are preferably was mutated at E6 positions 63 and 106 (based on SEQ ID NO:8). Mutants are created, for example, using the Quick Change Site-Directed Mutagenesis Kit (Stratagene, La Jolla, Calif.). HPV16 E6 containing a single point mutation in the codon for Cys¹⁰⁶ in SEQ ID NO:8 (=Cys 113 in SEQ ID NO:7). Cys¹⁰⁶ neither binds nor facilitates degradation of p53 and is incapable of immortalizing human mammary epithelial cells (MEC), a phenotype dependent upon p53 degradation. A single amino acid substitution at position Cys⁶³ of SEQ ID NO:8 (=Cys⁷⁰ in SEQ ID NO:7) destroys several HPV16 E6 functions: p53 degradation, E6TP-1 degradation, activation of telomerase, and, consequently, immortalization of primary epithelial cells.

Any nucleotide sequence that encodes this E6 polypeptide, or preferably, one of the mutants thereof discussed herein, or an antigenic or immunogenic fragment or epitope thereof, can be used in the present invention. Other mutations can be tested and used in accordance with the methods described herein including those described in Cassetti et al., supra. These mutations can be produced from any appropriate starting sequences by mutation of the coding DNA.

The present invention also includes the use of a tandem E6-E7 vaccine, using one or more of the mutations described herein to render the oncoproteins inactive with respect to their oncogenic potential in vivo. VRP vaccines (described in Cassetti et al., supra) include fused E6 and E7 genes in one open reading frame which are mutated at four or five amino acid positions. Thus, the present constructs may include one or more epitopes of E6 and E7, which may be arranged in their native order or shuffled in any way that permits the expressed protein to bear the E6 and E7 antigenic epitopes in an immunogenic form. DNA encoding amino acid spacers between E6 and E7 or between individual epitopes of these proteins may be introduced into the vector, provided again, that the spacers permit the expression or presentation of the epitopes in an immunogenic manner after they have been expressed by transduced host cells.

### DNA Encoding HPV L2

Wild type L2 nucleotide (SEQ ID NO:9) and amino acid (SEQ ID NO:10) sequences are shown below:

Wild type L2 nucleotide sequence obtained from HPV type 16 genomic DNA (see GenBank Accession No. K02718) includes the following sequence as SEQ ID NO: 9:

atgcga cacaaacgtt ctgcaaaacg cacaaaacgt gcatcggcta
cccaacttta taaaacatgc aaacaggcag gtacatgtcc
acctgacatt atacctaagg ttgaaggcaa aactattgct
gaacaaatat tacaatatgg aagtatgggt gtattttttg
gtgggttagg aattggaaca gggtcgggta caggcggacg
cactgggtat attccattgg gaacaaggcc tcccacagct
acagatacac ttgctcctgt aagaccccct ttaacagtag
atcctgtggg cccttctgat ccttctatag tttctttagt
ggaagaaact agttttattg atgctggtgc accaacatct
gtaccttcca ttcccccaga tgtatcagga tttagtatta
ctacttcaac tgataccaca cctgctatat tagatattaa
taatactgtt actactgtta ctacacataa taatcccact
ttcactgacc catctgtatt gcagcctcca acacctgcag
aaactggagg gcattttaca ctttcatcat ccactattag
tacacataat tatgaagaaa ttcctatgga tacatttatt
gttagcacaa accctaacac agtaactagt agcacaccca
taccagggtc tcgcccagtg gcacgcctag gattatatag
tcgcacaaca caacaggtta aagttgtaga ccctgctttt
gtaaccactc ccactaaact tattacatat gataatcctg
catatgaagg tatagatgtg gataatacat tatatttttc
tagtaatgat aatagtatta atatagctcc agatcctgac
tttttggata tagttgcttt acataggcca gcattaacct
ctaggcgtac tggcattagg tacagtagaa ttggtaataa
acaaacacta cgtactcgta gtggaaaatc tataggtgct
aaggtacatt attattatga tttaagtact attgatcctg
cagaagaaat agaattacaa actataacac cttctacata
tactaccact tcacatgcag cctcacctac ttctattaat
aatggattat atgatattta tgcagatgac tttattacag
atacttctac aaccccggta ccatctgtac cctctacatc
tttatcaggt tatattcctg caaatacaac aattcctttt
ggtggtgcat acaatattcc tttagtatca ggtcctgata
tacccattaa tataactgac caagctcctt cattaattcc
tatagttcca gggtctccac aatatacaat tattgctgat
gcaggtgact tttatttaca tcctagttat tacatgttac
gaaaacgacg taaacgttta ccatattttt tttcagatgt
ctctttggct gcctag

Wild type L2 amino acid sequence (see GenBank Accession Numbers AAD33258) includes the following sequence as SEQ ID NO: 10:

```
    1 mrhkrsakrt krasatqlyk tckqagtcpp diipkvegkt iadqilqygs mgvffgglgi
  61 gtgsgtggrt gyiplgtrpp tatdtlapvr ppltvdpvgp sdpsivslve etsfidagap
121 tsvpsippdv sgfsittstd ttpaildinn tvttvtthnn ptftdpsvlq pptpaetggh
181 ftlssstist hnyeeipmdt fivstnpntv tsstpipgsr pvarlglysr ttqqvkvvdp
241 afittptkli tydnpayegi dvdntlyfss ndnsiniapd pdfldivalh rpaltsrrtg
301 irysrignkq tlrtrsgksi gakvhyyydf stidsaeeie lqtitpstyt ttshaalpts
361 innglydiya ddfitdtstt pvpsvpstsl sgyipantti pfggaynipl vsgpdipini
421 tdqapslipi vpgspqytii adagdfylhp syymlrkrrk rlpyffsdvs laa
```

The present inventors and their colleagues have described HPV L2 nucleic acids and polypeptides, and fragments thereof. See e.g., PCT Publication No. WO2006/083984, and U.S. Pat. No. 6,599,739. Further, synthetic or recombinant L2 nucleic acids are provided herein. See, e.g., GenBank Accession No. AJ313180. Variant L1 and L2 genes are provided. See, e.g., GenBank Accession No. U37217.

### DNA Encoding Calreticulin (CRT)

The present inventors and their colleagues have described the use of calreticulin in DNA vaccines. See Cheng W F et al., Vaccine. 23:3864-74, 2005. As discussed herein and in the references, DNA vaccines encoding CRT linked either to E6 or to E7 generate significant antitumor effects against E6- and E7-expressing tumors, respectively. Moreover, simultaneous vaccination with both CRT/E6 and CRT/E7 DNA vaccines generated significant E6- and E7-specific T-cell immune responses and significantly better therapeutic antitumor effects against E6- and E7-expressing tumors than vaccination with either CRT/E6 DNA or CRT/E7 DNA alone.

The three domains of CRT also produce E7-specific antitumor immunity and antiangiogenic effects (Cheng W F et al., Vaccine. 23:3864-74, 2005). DNA vaccines encoding each of N, P, and C domains of CRT linked to E7 antigen produced significant stimulation of E7-specific CD8⁺ T cell precursors and antitumor effects against E7-expressing tumors. The N domain of CRT also showed antiangiogenic properties that might have contributed to the antitumor effect. Thus, the present invention includes DNA immunogens expressing the N, P, or C domain of CRT, or a combination thereof.

“Calreticulin” or “CRT” describes the well-characterized ˜46 kDa resident protein of the ER lumen that has lectin activity and participates in the folding and assembly of nascent glycoproteins. CRT acts as a “chaperone” polypeptide and a member of the MHC class I transporter TAP complex; CRT associates with TAP1 and TAP2 transporters, tapasin, MHC Class I heavy chain polypeptide and β2 microglobulin to function in the loading of peptide epitopes onto nascent MHC class I molecules (Jorgensen, Eur. J. Biochem. 267:2945-54, 2002). The term “calreticulin” or “CRT” refers to polypeptides and nucleic acids molecules having substantial identity (defined herein) to the exemplary CRT sequences as described herein. A CRT polypeptide is a polypeptides comprising a sequence identical to or substantially identical to the amino acid sequence of CRT. An exemplary nucleotide and amino acid sequence for a CRT used in the present compositions and methods are presented herein. The terms “calreticulin” or “CRT” encompass native proteins as well as recombinantly produced modified proteins that induce an immune response, including a CTL response. CRT encompasses homologues and allelic variants of CRT, including variants of native proteins constructed by in vitro techniques, and proteins isolated from natural sources. The CRT polypeptides of the invention, and sequences encoding them, also include fusion proteins including non-CRT sequences, particularly MHC class I-binding peptides; and also further including other domains, e.g., epitope tags, enzyme cleavage recognition sequences, signal sequences, secretion signals and the like.

The term “endoplasmic reticulum chaperone polypeptide” as used herein means any polypeptide having substantially the same ER chaperone function as the exemplary chaperone proteins CRT, tapasin, ER60 or calnexin. Thus, the term includes all functional fragments or variants or mimics thereof. A polypeptide or peptide can be routinely screened for its activity as an ER chaperone using assays known in the art, such as that set forth in Example 1 of U.S. patent application Ser. No. 11/773,162, filed Jul. 3, 2007. While the invention is not limited by any particular mechanism of action, in vivo chaperones promote the correct folding and oligomerization of many glycoproteins in the ER, including the assembly of the MHC class I heterotrimeric molecule (heavy (H) chain, β2m, and peptide). They also retain incompletely assembled MHC class I heterotrimeric complexes in the ER (Hauri FEBS Lett. 476:32-37, 2000).

The sequences of CRT, including human CRT, are well known in the art (McCauliffe, J. Clin. Invest. 86:332-5, 1990; Burns, Nature 367:476-80, 1994; Coppolino, Int. J. Biochem. Cell Biol 30:553-8, 2000). The nucleic acid sequence appears as GenBank Accession No. NM 004343 and is SEQ ID NO:11.

| 1 gtccgtactg cagagccgct gccggagggt cgttttaaag ggccgcgttg ccgccccctc | |
|---|---|
| 61 ggcccgccat gctgctatcc gtgccgctgc tgctcggcct cctcggcctg gccgtcgccg | |
| 121 agcccgccgt ctacttcaag gagcagtttc tggacggaga cgggtggact tcccgctgga | |
| 181 tcgaatccaa acacaagtca gattttggca aattcgttct cagttccggc aagttctacg | |
| 241 gtgacgagga gaaagataaa ggtttgcaga caagccagga tgcacgcttt tatgctctgt | |
| 301 cggccagttt cgagcctttc agcaacaaag gccagacgct ggtggtgcag ttcacggtga | |
| 361 aacatgagca gaacatcgac tgtgggggcg gctatgtgaa gctgtttcct aatagtttgg | |
| 421 accagacaga catgcacgga gactcagaat acaacatcat gtttggtccc gacatctgtg | |
| 481 gccctggcac caagaaggtt catgtcatct tcaactacaa gggcaagaac gtgctgatca | |
| 541 acaaggacat ccgttgcaag gatgatgagt ttacacacct gtacacactg attgtgcggc | |
| 601 cagacaacac ctatgaggtg aagattgaca acagccaggt ggagtccggc tccttggaag | |
| 661 acgattggga cttcctgcca cccaagaaga taaaggatcc tgatgcttca aaaccggaag | |
| 721 actgggatga gcgggccaag atcgatgatc ccacagactc caagcctgag gactgggaca | |
| 781 agcccgagca tatccctgac cctgatgcta agaagcccga ggactgggat gaagagatgg | |
| 841 acggagagtg ggaaccccca gtgattcaga accctgagta caagggtgag tggaagcccc | |
| 901 ggcagatcga caacccagat tacaagggca cttggatcca cccagaaatt gacaaccccg | |
| 961 agtattctcc cgatcccagt atctatgcct atgataactt tggcgtgctg ggcctggacc | |
| 1021 tctggcaggt caagtctggc accatctttg acaacttcct catcaccaac gatgaggcat | |
| 1081 acgctgagga gtttggcaac gagacgtggg gcgtaacaaa ggcagcagag aaacaaatga | |
| 1141 aggacaaaca ggacgaggag cagaggctta aggaggagga agaagacaag aaacgcaaag | |
| 1201 aggaggagga ggcagaggac aaggaggatg atgaggacaa agatgaggat gaggaggatg | |
| 1261 aggaggacaa ggaggaagat gaggaggaag atgtccccgg ccaggccaag gacgagctgt | |
| 1321 agagaggcct gcctccaggg ctggactgag gcctgagcgc tcctgccgca gagcttgccg | |
| 1381 cgccaaataa tgtctctgtg agactcgaga actttcattt ttttccaggc tggttcggat | |
| 1441 ttggggtgga ttttggtttt gttcccctcc tccactctcc cccaccccct ccccgccctt | |
| 1501 tttttttttt tttttaaact ggtattttat cctttgattc tccttcagcc ctcacccctg | |
| 1561 gttctcatct ttcttgatca acatcttttc ttgcctctgt gccccttctc tcatctctta | |
| 1621 gctcccctcc aacctggggg gcagtggtgt ggagaagcca caggcctgag atttcatctg | |
| 1681 ctctccttcc tggagcccag aggagggcag cagaaggggg tggtgtctcc aaccccccag | |
| 1741 cactgaggaa gaacggggct cttctcattt cacccctccc tttctcccct gcccccagga | |
| 1801 ctgggccact tctgggtggg gcagtgggtc ccagattggc tcacactgag aatgtaagaa | |
| 1861 ctacaaacaa aatttctatt aaattaaatt ttgtgtctc | 1899 |

Human CRT protein (GenBank Accession No. NM 004343), is shown below as SEQ ID NO: 12:

| 1 MLLSVPLLLG LLGLAVAEPA VYFKEQFLDG DGWTSRWIES KHKSDFGKFV LSSGKFYGDE | |
|---|---|
| 61 EKDKGLQTSQ DARFYALSAS FEPFSNKGQT LVVQFTVKHE QNIDCGGGYV KLFPNSLDQT | |
| 121 DMHGDSEYNI MFGPDICGPG TKKVHVIFNY KGKNVLINKD IRCKDDEFTH LYTLIVRPDN | |
| 181 TYEVKIDNSQ VESGSLEDDW DFLPPKKIKD PDASKPEDWD ERAKIDDPTD SKPEDWDKPE | |
| 241 HIPDPDAKKP EDWDEEMDGE WEPPVIQNPE YKGEWKPRQI DNPDYKGTWI HPEIDNPEYS | |
| 301 PDPSIYAYDN FGVLGLDLWQ VKSGTIFDNF LITNDEAYAE EFGNETWGVT KAAEKQMKDK | |
| 361 QDEEQRLKEE EEDKKRKEEE EAEDKEDDED KDEDEEDEED KEEDEEEDVP GQAKDEL | 417 |

### Nucleic Acids Encoding Calreticulin, E6 E7 and L2

Shown below as SEQ ID NO: 1 is a nucleic acid sequence of the invention that contains nucleic acid sequences encoding calreticulin (nucleotides 1-1251), E6 (1258-1682), E7 (1683-2025) and L2 (2032-2604). Additionally, SEQ ID NO: 1 contains two linker sequences (nucleotides 1252-1257 and 2026-2031).

| | |
|---|---|
| \| 10 \| 20 \| 30 \| 40 \| 50 \| 60 \| 70 \| 80 | |
| 1 atgctgctat ccgtgccgct gctgctcggc ctcctcggcc tggccgtcgc cgagcctgcc gtctacttca aggagcagtt | 80 |
| 81 tctggacggg gacgggtgga cttcccgctg gatcgaatcc aaacacaagt cagattttgg caaattcgtt ctcagttccg | 160 |
| 161 gcaagttcta cggtgacgag gagaaagata aaggtttgca gacaagccag gatgcacgct tttatgctct gtcggccagt | 240 |
| 241 ttcgagcctt tcagcaacaa aggccagacg ctggtggtgc agttcacggt gaaacatgag cagaacatcg actgtggggg | 320 |
| 321 cggctatgtg aagctgtttc ctaatagttt ggaccagaca gacatgcacg gagactcaga atacaacatc atgtttggtc | 400 |
| 401 ccgacatctg tggccctggc accaagaagg ttcatgtcat cttcaactac aagggcaaga acgtgctgat caacaaggac | 480 |
| 481 atccgttgca aggatgatga gtttacacac ctgtacacac tgattgtgcg gccagacaac acctatgagg tgaagattga | 560 |
| 561 caacagccag gtggagtccg gctccttgga agacgattgg gacttcctgc cacccaagaa gataaaggat cctgatgctt | 640 |
| 641 caaaaccgga agactgggat gagcgggcca agatcgatga tcccacagac tccaagcctg aggactggga caagcccgag | 720 |
| 721 catatccctg accctgatgc taagaagccc gaggactggg atgaagagat ggacggagag tgggaacccc cagtgattca | 800 |
| 801 gaaccctgag tacaagggtg agtggaagcc ccggcagatc gacaacccag attacaaggg cacttggatc cacccagaaa | 880 |
| 881 ttgacaaccc cgagtattct cccgatccca gtatctatgc ctatgataac tttggcgtgc tgggcctgga cctctggcag | 960 |
| 961 gtcaagtctg gcaccatctt tgacaacttc ctcatcacca acgatgaggc atacgctgag gagtttggca acgagacgtg | 1040 |
| 1041 gggcgtaaca aaggcagcag agaaacaaat gaaggacaaa caggacgagg agcagaggct taaggaggag gaagaagaca | 1120 |
| 1121 agaaacgcaa agaggaggag gaggcagagg acaaggagga tgatgaggac aaagatgagg atgaggagga tgaggaggac | 1200 |
| 1201 aaggaggaag atgaggagga agatgtcccc ggccaggcca aggacgagct gGAATTCATG CACCAGAAGA GAACCGCCAT | 1280 |
| 1281 GTTCCAGGAC CCCCAGGAGA GACCCAGGAA GCTGCCCCAG CTGTGCACCG AGCTGCAGAC CACCATCCAC GACATCATCC | 1360 |
| 1361 TGGAGTGCGT GTACTGCAAG CAGCAGCTGC TGAGGAGAGA GGTGTACGAC TTCGCCTTCC GGGACCTGTG CATCGTGTAC | 1440 |
| 1441 AGAGACGGCA ACCCCTACGC CGTGGGCGAC AAGTGCCTGA AGTTCTACAG CAAGATCAGC GAGTACAGAC ACTACTGCTA | 1520 |
| 1521 CAGCCTGTAC GGCACCACCC TGGAGCAGCA GTACAACAAG CCCCTGTGCG ACCTGCTGAT CCGGTGCATC AACGGCCAGA | 1600 |
| 1601 AGCCCCTGTG CCCCGAGGAG AAGCAGCGGC ACCTGGACAA GAAGCAGAGA TTCCACAACA TCAGGGGCCG GTGGACCGGC | 1680 |
| 1681 AGatgcatga gctgctgcag gagcagccgg accagacggg agacccagct gatgcacggc gacaccccca ccctgcacga | 1760 |
| 1761 gtacatgctg gacctgcagc cagagaccac cgacctgtac ggctacggcc agctgaacga cagcagcgag gaggaggacg | 1840 |
| 1841 agatcgacgg ccccgccggc caggccgagc ccgaccgggc ccactacaac atcgtgacct tctgctgcaa gtgcgacagc | 1920 |
| 1921 accctgaggc tgtgcgtgca gagcacccac gtggacatca gaaccctgga ggacctgctg atgggcaccc tgggcatcgt | 2000 |
| 2001 gtgccccatc tgcagccaga agcccTCTAG AATGAAGAGG GCCAGCGCCA CCCAGCTGTA CAAGACCTGC AAGCAGGCCG | 2080 |
| 2081 GCACCTGCCC CCCCGACATC ATCTCCAAGG TGGAGGGCAA GACCATCGCC GACCAGATCC TGCAGTACGG CAGCATGGGC | 2160 |
| 2161 GTGTTCTTCG GCGGCCTGGG CATCGGCACC GGCAGCGGCA CCGGCGGCAG GACCGGCTAC ATCCCCCTGG GCACCAGGCC | 2240 |
| 2241 CCCCACCGCC ACCGACACCC TGGCCCCCGT GAGGCCCCCC CTGACCGTGG ACCCCGTGGG CCCCAGCGAC CCCAGCATCG | 2320 |
| 2321 TGAGCCTGGT GGAGGAGACC AGCTTCATCG ACGCCGGCGC CCCCACCAGC GTGCCCAGCA TCCCCCCCGA CGTGAGCGGC | 2400 |
| 2401 TTCAGCATCA CCACCAGCAC CGACACCACC CCCGCCATCC TGGACATCAA CAACACCGTG ACCACCGTGA CCACCCACAA | 2480 |
| 2481 CAACCCCACC TTCACCGACC CCAGCGTGCT GCAGCCCCCC ACCCCCGCCG AGACCGGCGG CCACTTCACC CTGAGCAGCA | 2560 |
| 2561 GCACCATCAG CACCCACAAC TACGAGGAGA TCCCCATGGA CACC | 2604 |
| \| 10 \| 20 \| 30 \| 40 \| 50 \| 60 \| 70 \| 80 | |

### Polypeptide Antigens Contains Calreticulin, E6, E7 and L2

Shown below as SEQ ID NO: 2 is an amino acid sequence of the invention that contains nucleic acid sequences encoding calreticulin (amino acid residues 1-417), E6 (420-577), E7 (578-675) and L2 (678-868). Additionally, SEQ ID NO: 2 contains two amino acid linker peptides (amino acid residues 418-419 and 676-677).

| | |
|---|---|
| 10 \| 20 \| 30 \| 40 \| 50 \| 60 \| 70 \| 80 | |
| 1 mllsvplllg llglavaepa vyfkeqfldg dgwtsrwies khksdfgkfv lssgkfygde ekdkglqtsq darfyalsas | 80 |
| 81 fepfsnkgqt lvvqftvkhe qnidcgggyv klfpnsldqt dmhgdseyni mfgpdicgpg tkkvhvifny kgknvlinkd | 160 |
| 161 irckddefth lytlivrpdn tyevkidnsq vesgsleddw dflppkkikd pdaskpedwd erakiddptd skpedwdkpe | 240 |
| 241 hipdpdakkp edwdeemdge weppviqnpe ykgewkprqi dnpdykgtwi hpeidnpeys pdpsiyaydn fgvlgldlwq | 320 |
| 321 vksgtifdnf litndeayae efgnetwgvt kaaekqmkdk qdeeqrlkee eedkkrkeee eaedkedded kdedeedeed | 400 |
| 401 keedeeedvp gqakdelefM HQKRTAMFQD PQERPRKLPQ LCTELQTTIH DIILECVYCK QQLLRREVYD FAFRDLCIVY | 480 |
| 481 RDGNPYAVGD KCLKFYSKIS EYRHYCYSLY GTTLEQQYNK PLCDLLIRCI NGQKPLCPEE KQRHLDKKQR FHNIRGRWTG | 560 |
| 561 RCMSCCRSSR TRRETQLmhg dtptlheyml dlqpettdly gygqlndsse eedeidgpag qaepdrahyn ivtfcckcds | 640 |
| 641 tlrlcvqsth vdirtledll mgtlgivcpi csqkpSRMKR ASATQLYKTC KQAGTCPPDI ISKVEGKTIA DQILTYGSMG | 720 |
| 721 VFFGGLGIGT GSGTGGRTGY IPLGTRPPTA TDTLAPVRPP LTVDPVGPSD PSIVSLVEET SFIDAGAPTS VPSIPPDVSG | 800 |
| 801 FSITTSTDTT PAILDINNTV TTVTTHNNPT FTDPSVLQPP TPAETGGHFT LSSSTISTHN YEEEIPMDT | 868 |
| \| 10 \| 20 \| 30 \| 40 \| 50 \| 60 \| 70 \| 80 | |

Other Antigens Associated with Pathogens

A major use for the present invention is as a therapeutic vaccine for cancer and for major chronic viral infections that cause morbidity and mortality worldwide. Such vaccines are designed to eliminate infected cells, which requires a T cell response. The vaccines of the present invention are designed to meet these needs.

Preferred antigens are epitopes of pathogenic microorganisms against which the host is defended by effector T cells responses, including CTL and delayed type hypersensitivity. Thus, the types of antigens included in the vaccine compositions of this invention are any of those associated with such pathogens (in addition to tumor-specific antigens).

The two most common cancers worldwide, hepatoma and cervical cancer, are associated with viral infection. Hepatitis B virus (HBV) (Beasley, R. P. et al., Lancet 2:1129-1133 (1981) has been implicated as etiologic agent of hepatomas. 80-90% of cervical cancers express the E6 and E7 antigens (discussed above and exemplified herein) from one of four “high risk” human papillomavirus types: HPV-16, HPV-18, HPV-31 and HPV-45 (Gissmann, L. et al., Ciba Found Symp. 120:190-207, 1986; Beaudenon, S., et al. Nature 321:246-9, 1986). As described herein, the HPV E6 and E7 antigens are the most promising targets for virus associated cancers in immunocompetent individuals because of their ubiquitous expression in cervical cancer. In addition to their importance as targets for therapeutic cancer vaccines, virus associated tumor antigens are also ideal candidates for prophylactic vaccines. Indeed, introduction of prophylactic HBV vaccines in Asia have decreased the incidence of hepatoma (Chang, M H et al. New Engl. J. Med. 336, 1855-1859 (1997), representing a great impact on cancer prevention.

Additional viruses associated with chronic human viral infections are hepatitis C Virus (HCV), human immunodeficiency virus (HIV-1 and HIV-2), herpesviruses such as Epstein Barr Virus (EBV), cytomegalovirus (CMV) and HSV-1 and HSV-2, and influenza virus. Useful antigens include HBV surface antigen or HBV core antigen; ppUL83 or pp 89 of CMV; antigens of gp120, gp41 or p24 proteins of HIV-1; ICP27, gD2, gB of HSV; or influenza hemagglutinin or nucleoprotein (Anthony, L S et al., Vaccine 1999; 17:373-83). Other antigens associated with pathogens that can be utilized as described herein are antigens of various parasites, includes malaria, preferably malaria peptide based on repeats of NANP.

In addition to its applicability to human cancer and infectious diseases, the present invention is also intended for use in treating animal diseases in the veterinary medicine context. Thus, the approaches described herein may be readily applied by one skilled in the art to treatment of veterinary infections including equine herpesviruses, bovine viruses such as bovine viral diarrhea virus (for example, the E2 antigen), bovine herpesviruses, Marek's disease virus in chickens and other fowl; animal retroviral and lentiviral diseases (e.g., feline leukemia, feline immunodeficiency, simian immunodeficiency viruses, etc.); pseudorabies and rabies; and the like.

As for tumor antigens, any tumor-associated or tumor-specific antigen that can be recognized by T cells, preferably by CTL, can be used. These include, without limitation, mutant p53, HER2/neu or a peptide thereof, or any of a number of melanoma-associated antigens such as MAGE-1, MAGE-3, MART-1/Melan-A, tyrosinase, gp75, gp100, BAGE, GAGE-1, GAGE-2, GnT-V, and p15 (see, for example, U.S. Pat. No. 6,187,306).

### General Recombinant DNA Methods

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J D et al., Recombinant DNA, 2^{nd} Ed., Scientific American Books, New York, 1992; and Old, R W et al., Principles of Gene Manipulation: An Introduction to Genetic Engineering, 2nd Ed., University of California Press, Berkeley, Calif. (1981).

Techniques for the manipulation of nucleic acids, such as, e.g., generating mutations in sequences, subcloning, labeling probes, sequencing, hybridization and the like are well described in the scientific and patent literature. See, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immunofluorescence assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

### Amplification of Nucleic Acids

Oligonucleotide primers can be used to amplify nucleic acids to generate fusion protein coding sequences used to practice the invention, to monitor levels of vaccine after in vivo administration (e.g., levels of a plasmid or virus), to confirm the presence and phenotype of activated CTLs, and the like. The skilled artisan can select and design suitable oligonucleotide amplification primers using known sequences. Amplification methods are also well known in the art, and include, e.g., polymerase chain reaction, PCR (PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (Wu (1989) Genomics 4:560; Landegren (1988) Science 241:1077; Barringer (1990) Gene 89:117); transcription amplification (Kwoh (1989) Proc. Natl. Acad. Sci. USA 86:1173); and, self-sustained sequence replication (Guatelli (1990) Proc. Natl. Acad. Sci. USA 87:1874); Qβ replicase amplification (Smith (1997) J. Clin. Microbiol. 35:1477-1491; Burg (1996) Mol. Cell. Probes 10:257-271) and other RNA polymerase mediated techniques (NASBA, Cangene, Mississauga, Ontario; Berger (1987) Methods Enzymol. 152:307-316; U.S. Pats No. 4,683,195 and 4,683,202; Sooknanan (1995) Biotechnology 13:563-564).

Unless otherwise indicated, a particular nucleic acid sequence is intended to encompasses conservative substitution variants thereof (e.g., degenerate codon substitutions) and a complementary sequence. The term “nucleic acid” is synonymous with “polynucleotide” and is intended to include a gene, a cDNA molecule, an mRNA molecule, as well as a fragment of any of these such as an oligonucleotide, and further, equivalents thereof (explained more fully below). Sizes of nucleic acids are stated either as kilobases (kb) or base pairs (bp). These are estimates derived from agarose or polyacrylamide gel electrophoresis (PAGE), from nucleic acid sequences which are determined by the user or published. Protein size is stated as molecular mass in kilodaltons (kDa) or as length (number of amino acid residues). Protein size is estimated from PAGE, from sequencing, from presumptive amino acid sequences based on the coding nucleic acid sequence or from published amino acid sequences.

Specifically, cDNA molecules encoding the amino acid sequence corresponding to the fusion polypeptide of the present invention or fragments or derivatives thereof can be synthesized by the polymerase chain reaction (PCR) (see, for example, U.S. Pat. No. 4,683,202) using primers derived the sequence of the protein disclosed herein. These cDNA sequences can then be assembled into a eukaryotic or prokaryotic expression vector and the resulting vector can be used to direct the synthesis of the fusion polypeptide or its fragment or derivative by appropriate host cells, for example COS or CHO cells.

This invention includes isolated nucleic acids having a nucleotide sequence encoding the novel fusion polypeptides that comprise a translocation polypeptide and an antigen, fragments thereof or equivalents thereof. The term nucleic acid as used herein is intended to include such fragments or equivalents. The nucleic acid sequences of this invention can be DNA or RNA.

A cDNA nucleotide sequence the fusion polypeptide can be obtained by isolating total mRNA from an appropriate cell line. Double stranded cDNA is prepared from total mRNA. cDNA can be inserted into a suitable plasmid, bacteriophage or viral vector using any one of a number of known techniques.

In reference to a nucleotide sequence, the term “equivalent” is intended to include sequences encoding structurally homologous and/or a functionally equivalent proteins. For example, a natural polymorphism in a nucleotide sequence encoding an polypeptide according to the present invention (especially at the third base of a codon) may be manifest as “silent” mutations which do not change the amino acid sequence. Furthermore, there may be one or more naturally occurring isoforms or related, immunologically cross-reactive family members of these proteins. Such isoforms or family members are defined as proteins that share function amino acid sequence similarity to the reference polypeptide.

Fragment of Nucleic Acid

A fragment of the nucleic acid sequence is defined as a nucleotide sequence having fewer nucleotides than the nucleotide sequence encoding the full length translocation polypeptide, antigenic polypeptide or the fusion thereof. This invention includes such nucleic acid fragments that encode polypeptides which retain (1) the ability of the fusion polypeptide to induce increases in frequency or reactivity of T cells, preferably CD8+ T cells, that are specific for the antigen part of the fusion polypeptide.

An “immunogenically active fragment” or an “antigenic fragment” includes any portion of a protein or polypeptide described herein that functions as an antigen or an immunogen.

Nucleic acid sequences of this invention may also include linker sequences, natural or modified restriction endonuclease sites and other sequences that are useful for manipulations related to cloning, expression or purification of encoded protein or fragments. These and other modifications of nucleic acid sequences are described herein or are well-known in the art.

The techniques for assembling and expressing DNA coding sequences for translocation types of proteins, and DNA coding sequences for antigenic polypeptides, include synthesis of oligonucleotides, PCR, transforming cells, constructing vectors, expression systems, and the like; these are well-established in the art such that those of ordinary skill are familiar with standard resource materials, specific conditions and procedures.

### Expression Vectors and Host Cells

This invention includes an expression vector comprising a nucleic acid sequence encoding, which includes a promoter that is expressible in a eukaryotic cell, preferably in a mammalian cells, more preferably in a human cell.

The term “expression vector” or “expression cassette” as used herein refers to a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may also be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

Thus, expression cassettes include plasmids, recombinant viruses, any form of a recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include replicons (e.g., RNA replicons), bacteriophages) to which fragments of DNA may be attached and become replicated. Vectors thus include, but are not limited to RNA, autonomous self-replicating circular or linear DNA or RNA, e.g., plasmids, viruses, and the like (U.S. Pat. No. 5,217,879), and includes both the expression and nonexpression plasmids. Where a recombinant cell or culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Those skilled in the art appreciate that the particular design of an expression vector of this invention depends on considerations such as the host cell to be transfected and/or the type of protein to be expressed.

The present expression vectors comprise the full range of nucleic acid molecules encoding the various embodiments of the fusion polypeptide and its functional derivatives (defined herein) including polypeptide fragments, variants, etc.

Such expression vectors are used to transfect host cells (in vitro, ex vivo or in vivo) for expression of the DNA and production of the encoded proteins which include fusion proteins or peptides. It will be understood that a genetically modified cell expressing the fusion polypeptide may transiently express the exogenous DNA for a time sufficient for the cell to be useful for its stated purpose.

The present invention provides methods for producing the fusion polypeptides, fragments and derivatives. For example, a host cell transfected with a nucleic acid vector that encodes the fusion polypeptide or an siRNA is cultured under appropriate conditions to allow expression of the polypeptide.

Host cells may also be transfected with one or more expression vectors that singly or in combination include (a) DNA encoding at least a portion of the fusion polypeptide and (b) DNA encoding at least a portion of a second protein, so that the host cells produce yet further fusion polypeptides.

A culture typically includes host cells, appropriate growth media and other byproducts. Suitable culture media are well known in the art. The fusion polypeptide can be isolated from medium or cell lysates using conventional techniques for purifying proteins and peptides, including ammonium sulfate precipitation, fractionation column chromatography (e.g. ion exchange, gel filtration, affinity chromatography, etc.) and/or electrophoresis (see generally, “Enzyme Purification and Related Techniques”, Meth Enzymol, 22:233-577 (1971)). Once purified, partially or to homogeneity, the recombinant polypeptides or siRNAs of the invention can be utilized in pharmaceutical compositions as described in more detail herein.

The term “isolated” as used herein, when referring to a molecule or composition, such as a translocation polypeptide or a nucleic acid coding therefor, means that the molecule or composition is separated from at least one other compound (protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). Even where a protein has been isolated so as to appear as a homogenous or dominant band in a gel pattern, there are trace contaminants which co-purify with it.

Host cells transformed or transfected to express the fusion polypeptide or a homologue or functional derivative thereof are within the scope of the invention. For example, the fusion polypeptide may be expressed in yeast, or mammalian cells such as Chinese hamster ovary cells (CHO) or, preferably human cells. Preferred cells for expression of the proteins of the present invention are APCs most preferably, DCs. Other suitable host cells are known to those skilled in the art.

Expression in eukaryotic cells leads to partial or complete glycosylation and/or formation of relevant inter- or intra-chain disulfide bonds of the recombinant protein.

Although preferred vectors are described in the Examples, other examples of expression vectors are provided here. Examples of vectors for expression in yeast S. cerevisiae include pYepSec1 (Baldari et al., EMBO J. 6:229-34, 1987), pMFa (Kurjan et al., Cell 30:933-43, 1982), pJRY88 (Schultz et al., Gene 54:113-23, 1987), and pYES2 (Invitrogen Corp.). Baculovirus vectors available for expression of proteins in cultured insect cells (SF 9 cells) include the pAc series (Smith et al., Mol. Cell Biol. 3:2156-65, 1983) and the pVL series (Lucklow, V A et al., Virology 170:31-9, 1989). Generally, COS cells (Gluzman, Y., Cell 23:175-82, 1981) are used in conjunction with such vectors as pCDM 8 (Aruffo A et al., supra, for transient amplification/expression in mammalian cells, while CHO (dhfr-negative CHO) cells are used with vectors such as pMT2PC (Kaufman et al. EMBO J. 6:187-95, 1987) for stable amplification/expression in mammalian cells. The NS0 myeloma cell line (a glutamine synthetase expression system) is available from Celltech Ltd.

Often, in fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase. Typical fusion expression vectors include pGEX (Amrad Corp., Melbourne, Australia), pMAL (New England Biolabs) and pRIT5 (Pharmacia, Piscataway, N.J.) which fuse glutathione S-transferase, maltose E binding protein, or protein A, respectively, to the target recombinant protein. Inducible non-fusion expression vectors include pTrc (Amann et al., Gene 69:301-15, 1988) and pET 11d (Studier et al., Gene Expression Technology: Meth Enzymol 185:60-89, Academic Press, 1990).

### Vector Construction

Construction of suitable vectors comprising the desired coding and control sequences employs standard ligation and restriction techniques which are well understood in the art. Isolated plasmids, DNA sequences, or synthesized oligonucleotides are cleaved, tailored, and re-ligated in the form desired. The sequences of several preferred plasmid vectors, with and without inserted coding sequences, have been disclosed herein.

The DNA sequences which form the vectors are available from a number of sources. Backbone vectors and control systems are generally found on available “host” vectors which are used for the bulk of the sequences in construction. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleotide derivatives. The entire gene sequence for genes of sizeable length, e.g., 500-100,000 bp may be prepared by synthesizing individual overlapping complementary oligonucleotides and filling in single stranded nonoverlapping portions using DNA polymerase in the presence of the deoxyribonucleotide triphosphates. This approach has been used successfully in the construction of several genes of known sequence. (See, for example, Edge, M D, Nature 292:756, 1981; Nambair, K P, et al., Science 223:1299, 1984; Jay, E, J Biol Chem 259:6311, 1984).

Synthetic oligonucleotides are prepared by either the phosphotriester method as described by references cited above or the phosphoramidite method (Beaucage, S L et al., Tet Lett 22:1859, 1981; Matteucci, M D et al., J Am Chem Soc 103:3185, 1981) and can be prepared using commercially available automated oligonucleotide synthesizers. Kinase treatment of single strands prior to annealing or for labeling is by conventional methods.

Once the components of the desired vectors are thus available, they can be excised and ligated using standard restriction and ligation procedures. Site-specific DNA cleavage is performed by treating with the suitable restriction enzyme (or enzymes) under conditions which are generally understood in the art, and the particulars of which are specified by the manufacturer of these commercially available restriction enzymes. See, e.g., New England Biolabs, Product Catalog. A general description of size separations is found in Methods in Enzymology (1980) 65:499-560.

Restriction cleaved fragments may be blunt ended by treating with the large fragment of E. coli DNA polymerase I (Klenow) in the presence of the four deoxynucleotide triphosphates (dNTPs) using conventional methods and conditions. Ligations are performed using conventional methods. In vector construction employing “vector fragments”, the fragment is commonly treated with bacterial or mammalian alkaline phosphatase to remove the 5′ phosphate and prevent self-ligation. Alternatively, re-ligation can be prevented in vectors which have been double digested by additional restriction enzyme and separation of the unwanted fragments.

Any of a number of methods are used to introduce mutations into the coding sequence to generate the variants of the invention. These mutations include simple deletions or insertions, systematic deletions, insertions or substitutions of clusters of bases or substitutions of single bases.

For example, modifications of DNA sequences are created by site-directed mutagenesis, a well-known technique for which protocols and reagents are commercially available (Zoller, M J et al., Nucleic Acids Res 10:6487-500, 1982; Adelman, J P et al., DNA 2:183-193, 1983). Using conventional methods, transformants are selected based on the presence of the ampicillin-, tetracycline-, or other antibiotic resistance gene (or other selectable marker) depending on the mode of plasmid construction. Plasmids are then prepared from the transformants with optional chloramphenicol amplification (Clewell, D B et al., Proc Natl Acad Sci USA 62:1159, 1969); Clewell, D B, J Bacteriol 110:667, 1969)). Several mini DNA preps are commonly used. (See, e.g., Anal Biochem 114:193-7, 1981; Nucleic Acids Res 7:1513-23, 1979). The isolated DNA is analyzed by restriction and/or sequenced by the dideoxy nucleotide method of Sanger (Proc Natl Acad Sci USA 74:5463, 1977; Messing, et al., Nucleic Acids Res 9:309, 1981), or by the method of Maxam et al., Meth Enzymology 65:499, 1980.

Vector DNA can be introduced into mammalian cells via conventional techniques such as calcium phosphate or calcium chloride co-precipitation, DEAE-dextran-mediated transfection, lipofection, or electroporation. Suitable methods for transforming host cells can be found in Sambrook et al. supra and other standard texts. In fusion expression vectors, a proteolytic cleavage site may be introduced at the junction of two sequences (such as a reporter group and the target protein to enable separation of the target protein from the reporter group subsequent to purification of the fusion protein). Proteolytic enzymes for such cleavage and their recognition sequences include Factor Xa, thrombin and enterokinase.

### Promoters and Enhancers

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

The preferred promoter sequences of the present invention must be operable in mammalian cells and may be either eukaryotic or viral promoters. Although preferred promoters are described in the Examples, other useful promoters and regulatory elements are discussed below. Suitable promoters may be inducible, repressible or constitutive. A “constitutive” promoter is one which is active under most conditions encountered in the cell's environmental and throughout development. An “inducible” promoter is one which is under environmental or developmental regulation. A “tissue specific” promoter is active in certain tissue types of an organism. An example of a constitutive promoter is the viral promoter MSV-LTR, which is efficient and active in a variety of cell types, and, in contrast to most other promoters, has the same enhancing activity in arrested and growing cells. Other preferred viral promoters include that present in the CMV-LTR (from cytomegalovirus) (Bashart, M. et al., Cell 41:521, 1985) or in the RSV-LTR (from Rous sarcoma virus) (Gorman, C. M., Proc. Natl. Acad. Sci. USA 79:6777, 1982). Also useful are the promoter of the mouse metallothionein I gene (Hamer, D, et al., J. Mol. Appl. Gen. 1:273-88, 1982; the TK promoter of Herpes virus (McKnight, S, Cell 31:355-65, 1982); the SV40 early promoter (Benoist, C., et al., Nature 290:304-10, 1981); and the yeast gal4 gene promoter (Johnston, S A et al., Proc. Natl. Acad. Sci. USA 79:6971-5, 1982); Silver, P A, et al., Proc. Natl. Acad. Sci. (USA) 81:5951-5, 1984)). Other illustrative descriptions of transcriptional factor association with promoter regions and the separate activation and DNA binding of transcription factors include: Keegan et al., Nature 231:699, 1986; Fields et al., Nature 340:245, 1989; Jones, Cell 61:9, 1990; Lewin, Cell 61:1161, 1990; Ptashne et al., Nature 346:329, 1990; Adams et al., Cell 72:306, 1993. The relevant disclosure of all of these above-listed references is hereby incorporated by reference.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed in (Roy-Burman et al., U.S. Pat. No. 5,112,767). For a general discussion of enhancers and their actions in transcription, see, Lewin, B M, Genes IV, Oxford University Press pp. 552-576, 1990 (or later edition). Particularly useful are retroviral enhancers (e.g., viral LTR) that is preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

Nucleic acids of the invention can also be chemically synthesized using standard techniques, including solid-phase synthesis which, like peptide synthesis, has been fully automated with commercially available DNA synthesizers (Itakura U.S. Pats. No. 4,598,049, 4,401,796 and 4,373,071; Caruthers et al. U.S. Pat. No. 4,458,066.

### Proteins and Polypeptides

The terms “polypeptide,” “protein,” and “peptide” when referring to compositions of the invention are meant to include variants, analogues, and mimetics with structures and/or activity that substantially correspond to the polypeptide or peptide from which the variant, etc., was derived.

The present invention includes an “isolated” fusion polypeptide comprising a targeting polypeptide linked to an antigenic polypeptide.

The term “chimeric” or “fusion” polypeptide or protein refers to a composition comprising at least one polypeptide or peptide sequence or domain that is chemically bound in a linear fashion with a second polypeptide or peptide domain. One embodiment of this invention is an isolated or recombinant nucleic acid molecule encoding a fusion protein. The “fusion” can be an association generated by a peptide bond, a chemical linking, a charge interaction (e.g., electrostatic attractions, such as salt bridges, H-bonding, etc.) or the like. If the polypeptides are recombinant, the “fusion protein” can be translated from a common mRNA. Alternatively, the compositions of the domains can be linked by any chemical or electrostatic means. The chimeric molecules of the invention (e.g., targeting polypeptide fusion proteins) can also include additional sequences, e.g., linkers, epitope tags, enzyme cleavage recognition sequences, signal sequences, secretion signals, and the like. Alternatively, a peptide can be linked to a carrier simply to facilitate manipulation or identification/location of the peptide.

Also included is a “functional derivative” of protein which refers to an amino acid substitution variant, a “fragment,” or a “chemical derivative” of the protein, which terms are defined herein. A functional derivative of a protein retains measurable immunogenic activity, preferably that is manifest as promoting immunogenicity of one or more antigenic epitopes fused thereto or co-administered therewith. “Functional derivatives” encompass “variants” and “fragments” regardless of whether the terms are used in the conjunctive or the alternative herein.

A functional homologue must possess the above biochemical and biological activity. In view of this functional characterization, use of homologous proteins including proteins not yet discovered, fall within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, or 90% of the length of the reference sequence. The amino acid residues (or nucleotides) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to a reference nucleic acid molecules. BLAST protein searches can be performed with the XBLAST program, score=50, wordlength=3 to obtain amino acid sequences homologous to HPV22 protein molecules. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Thus, a homologue of a particular protein as described herein is characterized as having sequence similarity to a native protein when determined as above, of at least about 20% (at the amino acid level), preferably at least about 40%, more preferably at least about 70%, even more preferably at least about 90%, 95%, 97%, 98% or 99%.

It is within the skill in the art to obtain and express such a protein using DNA probes based on the disclosed sequences.

Then, the chimeric DNA construct or fusion protein's biological activity can be tested readily using art-recognized methods such as those described herein. A biological assay of the stimulation of antigen-specific T cell reactivity will indicate whether the homologue has the requisite activity to qualify as a “functional” homologue.

A “variant” of a protein refers to a molecule substantially identical to either the full protein or to a fragment thereof in which one or more amino acid residues have been replaced (substitution variant) or which has one or several residues deleted (deletion variant) or added (addition variant). A “fragment” of the protein refers to any subset of the molecule, that is, a shorter polypeptide of the full-length protein.

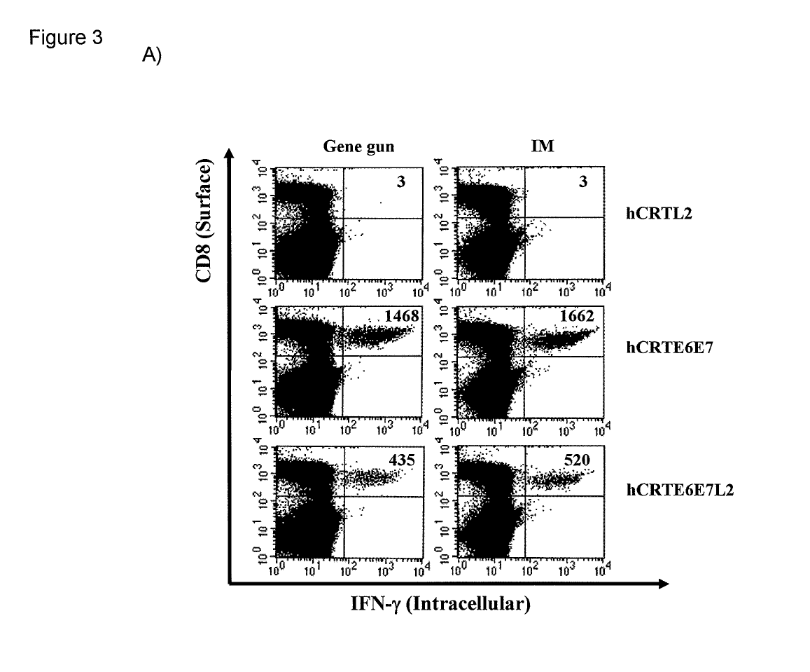

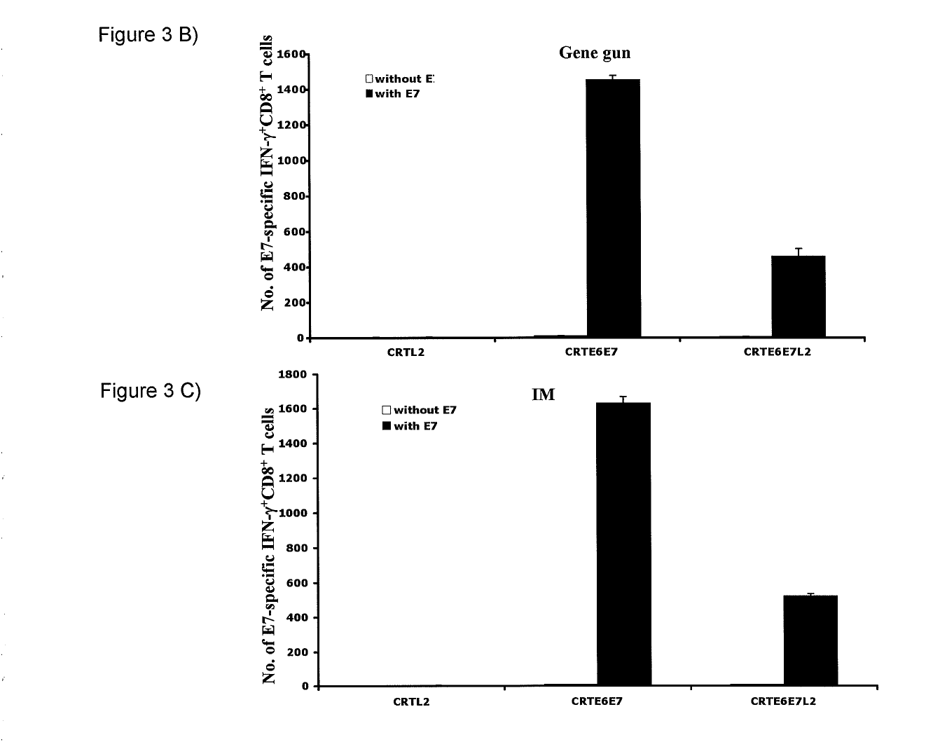

A preferred group of conservative variants are those in which at least one amino acid residue and preferably, only one, has been substituted by different residue. For a detailed description of protein chemistry and structure, see Schulz, G E et al., Principles of Protein Structure, Springer-Verlag, New York, 1978, and Creighton, T. E., Proteins: Structure and Molecular Properties, W. H. Freeman & Co., San Francisco, 1983, which are hereby incorporated by reference. The types of substitutions that may be made in the protein molecule may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

| | | |
|---|---|---|
| 1 | Small aliphatic, nonpolar or slightly | Ala, Ser, Thr (Pro, Gly); |
| | polar residues | |
| 2 | Polar, negatively charged residues and | Asp, Asn, Glu, Gln; |
| | their amides | |
| 3 | Polar, positively charged residues | His, Arg, Lys; |
| 4 | Large aliphatic, nonpolar residues | Met, Leu, Ile, Val (Cys) |
| 5 | Large aromatic residues | Phe, Tyr, Trp. |

The three amino acid residues in parentheses above have special roles in protein architecture. Gly is the only residue lacking a side chain and thus imparts flexibility to the chain. Pro, because of its unusual geometry, tightly constrains the chain. Cys can participate in disulfide bond formation, which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those that do not produce radical changes in the characteristics of the wild-type or native proteins. However, when it is difficult to predict the exact effect of the substitution, deletion or insertion in advance of doing so, one skilled in the art will appreciate that the effect can be evaluated by routine screening assays such as those described here, without requiring undue experimentation.

The term “chemically linked” refers to any chemical bonding of two moieties, e.g., as in one embodiment of the invention, where a translocation polypeptide is chemically linked to an antigenic peptide. Such chemical linking includes the peptide bonds of a recombinantly or in vivo generated fusion protein.

Therapeutic Compositions and their Administration

A vaccine composition including the nucleic acid encoding the antigen or the antigen in a fusion polypeptide, a particle comprising the nucleic acid or a cell expressing this nucleic acid, is administered to a mammalian subject, preferably a human. Another embodiment is a vaccine composition comprising DCs that are loaded with the antigen. The vaccine composition or the modified DCs are administered in a pharmaceutically acceptable carrier in a biologically effective or a therapeutically effective amount.

Certain preferred conditions are disclosed in the Examples. The composition may be given alone or in combination with another protein or peptide such as an immunostimulatory molecule. Treatment may include administration of an adjuvant, used in its broadest sense to include any nonspecific immune stimulating compound, such as an interferon. Adjuvants contemplated herein include resorcinols, non-ionic surfactants such as polyoxyethylene oleyl ether and n-hexadecyl polyethylene ether.

A therapeutically effective amount is a dosage that, when given for an effective period of time, achieves the desired immunological or clinical effect.

A therapeutically active amount of a nucleic acid encoding the fusion polypeptide may vary according to factors such as the disease state, age, sex, and weight of the individual, and the ability of the peptide to elicit a desired response in the individual. Dosage regimes may be adjusted to provide the optimum therapeutic response. For example, several divided doses may be administered daily or the dose may be proportionally reduced as indicated by the exigencies of the therapeutic situation. A therapeutically effective amounts of the protein, in cell associated form may be stated in terms of the protein or cell equivalents.

Thus an effective amount of the vaccine is between about 1 nanogram and about 1 gram per kilogram of body weight of the recipient, more preferably between about 0.1 μg/kg and about 10 mg/kg, more preferably between about 1 μg/kg and about 1 mg/kg. Dosage forms suitable for internal administration preferably contain (for the latter dose range) from about 0.1 μg to 100 μg of active ingredient per unit. The active ingredient may vary from 0.5 to 95% by weight based on the total weight of the composition. Alternatively, an effective dose of DCs loaded with the antigen is between about 10⁴ and 10⁸ cells. Those skilled in the art of immunotherapy will be able to adjust these doses without undue experimentation.

The composition may be administered in a convenient manner, e.g., injection by a convenient and effective route. Preferred routes for the DNA include intradermal “gene gun” delivery or intramuscular injection. The modified DCs are preferably administered by subcutaneous, intravenous or intramuscular routes. Other possible routes include oral administration, intrathecal, inhalation, transdermal application, or rectal administration. For the treatment of existing tumors which have not been completely resected or which have recurred, direct intratumoral injection is also intended.

Depending on the route of administration, the composition may be coated in a material to protect the compound from the action of enzymes, acids and other natural conditions which may inactivate the compound. Thus it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol) or in an appropriate carrier such as liposomes (including water-in-oil-in-water emulsions as well as conventional liposomes (Strejan et al., J. Neuroimmunol 7:27, 1984).

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Preferred pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the composition should be sterile and should be fluid. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms in the pharmaceutical composition can be achieved by various antibacterial and antifungal agents, for example, parabens, chlorobutanol, phenol, ascorbic acid, thimerosal, and the like.

Compositions are preferably formulated in dosage unit form for ease of administration and uniformity of dosage. Dosage unit form refers to physically discrete units suited as unitary dosages for a mammalian subject; each unit contains a predetermined quantity of active material (e.g., the nucleic acid vaccine) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects

For lung instillation, aerosolized solutions are used. In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

Other pharmaceutically acceptable carriers for the nucleic acid vaccine compositions according to the present invention are liposomes, pharmaceutical compositions in which the active protein is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

### Delivery of Vaccine Nucleic Acid to Cells and Animals

The following references set forth principles and current information in the field of basic, medical and veterinary virology and are incorporated by reference: Fields Virology, Fields, B N et al., eds., Lippincott Williams & Wilkins, NY, 1996; Principles of Virology: Molecular Biology, Pathogenesis, and Control, Flint, S. J. et al., eds., Amer Soc Microbiol, Washington D.C., 1999; Principles and Practice of Clinical Virology, 4th Edition, Zuckerman. A. J. et al., eds, John Wiley & Sons, NY, 1999; The Hepatitis C Viruses, by Hagedorn, C H et al., eds., Springer Verlag, 1999; Hepatitis B Virus: Molecular Mechanisms in Disease and Novel Strategies for Therapy, Koshy, R. et al., eds, World Scientific Pub Co, 1998; Veterinary Virology, Murphy, F. A. et al., eds., Academic Press, NY, 1999; Avian Viruses: Function and Control, Ritchie, B. W., Iowa State University Press, Ames, 2000; Virus Taxonomy: Classification and Nomenclature of Viruses: Seventh Report of the International Committee on Taxonomy of Viruses, by M. H. V. Van Regenmortel, M H V et al., eds., Academic Press; NY, 2000.

The Examples below describe certain preferred approaches to delivery of the vaccines and combinations of the present invention. A broader description of other approaches including viral and nonviral vectors and delivery mechanisms follow.

DNA delivery involves introduction of a “foreign” DNA into a cell ex vivo and ultimately, into a live animal or directly into the animal. Several general strategies for gene delivery (=delivery of nucleic acid vectors) for purposes that include “gene therapy” have been studied and reviewed extensively (Yang, N-S., Crit. Rev. Biotechnol. 12:335-356 (1992); Anderson, W F, Science 256:808-13, 1992; Miller, A S, Nature 357:455-60, 1992; Crystal, R G, Amer. J. Med. 92(suppl 6A):44-52S, 1992; Zwiebel, J A et al., Ann NY Acad Sc. 618:394-404, 1991; McLachlin, J R et al., Prog. Nucl. Acid Res. Molec. Biol. 38:91-135, 1990; Kohn, D B et al., Cancer Invest. 7:179-92, 1989), which references are herein incorporated by reference in their entirety).

One approach comprises nucleic acid transfer into primary cells in culture followed by autologous transplantation of the ex vivo transformed cells into the host, either systemically or into a particular organ or tissue.

The term “systemic administration” refers to administration of a composition or agent such as a molecular vaccine as described herein, in a manner that results in the introduction of the composition into the subject's circulatory system or otherwise permits its spread throughout the body. “Regional” administration refers to administration into a specific, and somewhat more limited, anatomical space, such as intraperitoneal, intrathecal, subdural, or to a specific organ. The term “local administration” refers to administration of a composition or drug into a limited, or circumscribed, anatomic space, such as intratumoral injection into a tumor mass, subcutaneous injections, intramuscular injections. One of skill in the art would understand that local administration or regional administration may also result in entry of a composition into the circulatory system.

For accomplishing the objectives of the present invention, nucleic acid therapy would be accomplished by direct transfer of a the functionally active DNA into mammalian somatic tissue or organ in vivo. DNA transfer can be achieved using a number of approaches described herein. These systems can be tested for successful expression in vitro by use of a selectable marker (e.g., G418 resistance) to select transfected clones expressing the DNA, followed by detection of the presence of the antigen-containing expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

The DNA molecules encoding the fusion polypeptides of the present invention may be packaged into retrovirus vectors using packaging cell lines that produce replication-defective retroviruses, as is well-known in the art (see, for example, Cone, R. D. et al., Proc. Natl. Acad. Sci. USA 81:6349-6353 (1984); Mann, R. F. et al., Cell 33:153-159 (1983); Miller, A. D. et al., Molec. Cell Biol. 5:431-437 (1985); Sorge, J., et al., Molec. Cell. Biol. 4:1730-1737 (1984); Hock, R. A. et al., Nature 320:257 (1986); Miller, A. D. et al., Molec. Cell. Biol. 6:2895-2902 (1986). Newer packaging cell lines which are efficient an safe for gene transfer have also been described (Bank et al., U.S. Pat. No. 5,278,056.

This approach can be utilized in a site specific manner to deliver the retroviral vector to the tissue or organ of choice. Thus, for example, a catheter delivery system can be used (Nabel, E G et al., Science 244:1342 (1989)). Such methods, using either a retroviral vector or a liposome vector, are particularly useful to deliver the nucleic acid to be expressed to a blood vessel wall, or into the blood circulation of a tumor.

Other virus vectors may also be used, including recombinant adenoviruses (Horowitz, M S, In: Virology, Fields, B N et al., eds, Raven Press, NY, 1990, p. 1679; Berkner, K L, Biotechniques 6:616-29, 1988; Strauss, S E, In: The Adenoviruses, Ginsberg, H S, ed., Plenum Press, NY, 1984, chapter 11), herpes simplex virus (HSV) for neuron-specific delivery and persistence. Advantages of adenovirus vectors for human gene delivery include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R J et al., EMBO J. 10:3941, 1991) according to the present invention.

Another vector which can express the DNA molecule of the present invention, and is useful in the present therapeutic setting is vaccinia virus, which can be rendered non-replicating (U.S. Pats. 5,225,336; 5,204,243; 5,155,020; 4,769,330; Sutter, G et al., Proc Natl Acad Sci USA 89:10847-51, 1992; Fuerst, T R et al., Proc. Natl. Acad. Sci. USA 86:2549-53, 1992; Falkner F G et al.; Nucl. Acids Res 15:7192, 1987; Chakrabarti, S et al., Mol Cell Biol 5:3403-9, 1985). Descriptions of recombinant vaccinia viruses and other viruses containing heterologous DNA and their uses in immunization and DNA therapy are reviewed in: Moss, B, Curr Opin Genet Dev 3:86-90, 1993; Moss, B, Biotechnol. 20:345-62, 1992); Moss, B, Curr Top Microbiol Immunol 158:25-38, 1992; Moss, B, Science 252:1662-7, 1991; Piccini, A et al., Adv. Virus Res 34:43-64, 1988; Moss, B et al., Gene Amplif Anal 3:201-13, 1983).

In addition to naked DNA or RNA, or viral vectors, engineered bacteria may be used as vectors. A number of bacterial strains including Salmonella, BCG and Listeria monocytogenes(LM) (Hoiseth et al., Nature 291:238-239, 1981; Poirier, T P et al., J. Exp. Med. 168:25-32, 1988); Sadoff, J C et al., Science 240:336-8, 1988; Stover, C K et al., Nature 351:456-60, 1991; Aldovini, A et al., Nature 351:479-82, 1991; Schafer, R, et al., J Immunol 149:53-9 (1992); Ikonomidis, G et al., J Exp Med 180 :2209-18, 1994). These organisms display two promising characteristics for use as vaccine vectors: (1) enteric routes of infection, providing the possibility of oral vaccine delivery; and (2) infection of monocytes/macrophages thereby targeting antigens to professional APCs.

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N-S, et al., Proc Natl Acad Sci USA 87:9568, 1990; Williams, R S et al., Proc Natl Acad Sci USA 88:2726, 1991; Zelenin, A V et al., FEBS Lett 280:94, 1991; Zelenin, A V et al., FEBS Lett 244:65, 1989); Johnston, S A et al., In Vitro Cell Dev Biol 27:11, 1991). Furthermore, electroporation, a well-known means to transfer genes into cell in vitro, can be used to transfer DNA molecules according to the present invention to tissues in vivo (Titomirov, A V et al., Biochim Biophys Acta 1088:131, 1991).

“Carrier mediated gene transfer” has also been described (Wu, C H et al., J Biol Chem 264:16985, 1989; Wu, G Y et al., J Biol Chem 263:14621, 1988; Soriano, P et al., Proc Nat. Acad Sci USA 80:7128, 1983; Wang, C-Y et al., Pro. Natl Acad Sci USA 84:7851, 1982; Wilson, J M et al., J Biol Chem 267:963, 1992). Preferred carriers are targeted liposomes (Nicolau, C et al., Proc Natl Acad Sci USA 80:1068, 1983; Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a target tissue-recognizing molecule (e.g., asialo-orosomucoid for liver) and a DNA binding compound to bind to the DNA to be transfected without causing damage, such as polylysine. This conjugate is then complexed with plasmid DNA of the present invention.

Plasmid DNA used for transfection or microinjection may be prepared using methods well-known in the art, for example using the Quiagen procedure (Quiagen), followed by DNA purification using known methods, such as the methods exemplified herein.

Having now generally described the invention, the same will be more readily understood through reference to the following examples which are provided by way of illustration, and are not intended to be limiting of the present invention, unless specified.

### EXAMPLE 1

### Generation and Characterization of a Preventive and Therapeutic HPV DNA Vaccine

This example corresponds to the experiments described in Kim et al., Vaccine (2008) 26(3): 351-60, the contents of which are hereby incorporated by reference in their entirety.

Here, an HPV DNA vaccine is described encoding calreticulin linked to HPV16 early proteins E6 and E7 and the late protein L2 (herein “hCRTE6E7L2”). We found that vaccination with hCRTE6E7L2 DNA vaccine induced a potent E6/E7-specific CD8+ T cell immune response and resulted in a significant therapeutic effect against E6/E7 expressing tumor cells. Furthermore, vaccination with hCRTE6E7L2 generated significant L2-specific neutralizing antibody responses against HPV-16 pseudovirion infection. The hCRTE6E7L2 DNA vaccines generate potent preventive and therapeutic effects in vaccinated mice.

Materials and Methods

### Plasmid DNA Constructs

To generate pNGVL4a-hCRTE6E7L2, L2 was first amplified with PCR using pET-28a-HPV 16L2 (11-200) as a template and a set of primers, 5′-aaatctagaatgaagagggccagcgccaa-3′ and 5′-aaagcggccgctcaggtgtccatgggatctc-3′. The 11-200aa L2 DNA was amplified from codon modified full length L2 and further cloned into pET28a vector. The amplified product was then cloned into the XbaI/NotI sites of pNGVL4a vector (National Gene Vector Laboratory). hCRT was PCR amplified by primers 5′-aaagtcgacatgctgctatccgtgccgctgc-3′ and 5′-gaattcgttgtctggccgcacaatca-3′ using a human CRT plasmid as a template. The PCR product was cut with Sal I/Eco RI and cloned into the Sal I/Eco RI sites of pNGVL4a-L2. HPV-16 E6 and E7 were codon optimized to enhance the translation of these genes. Codon optimized HPV-16 E6 and E7 was synthesized by GenScript Corporation (Piscataway, N.J.) was cloned into EcoRI/XbaI sites of pNGVL4a-hCRTL2. The promoter driving the expression of the pNGVL4a vector is CMV. For generation of pNGVL4a-hCRTL2, L2 was amplified again with another set of primers, 5′-tttgaattcatgaagagggccagcgcca-3′ and 5′-tttagatcttcaggtgtccatggggatct-3′. The PCR product was cut with EcoRI/BglII and cloned into the EcoRI/BglII sites of pNGVL4a-hCRT. The accuracy of DNA constructs was confirmed by DNA sequencing. The various genes were amplified and cloned such that each subsequent gene was “in frame” and stop codons were removed.

### Mice and Cell Lines

Female C57BL/6 mice (6 to 8 weeks old) were purchased from the National Cancer Institute (Frederick, Md, USA) and kept in the animal facility of the Johns Hopkins Hospital (Baltimore, Md, USA). All animal procedures were performed according to approved protocols and in accordance with the recommendations for the proper use and care of laboratory animals. The production and maintenance of TC-1 cells has been described.

### Western Blot Analysis.

BHK21 cells (ATCC, 2×10⁶) were transiently transfected with 2 μg of each plasmid construct using Lipofectamine 2000 (Invitrogen) according to the vendor's manual. Cells were grown at 37° C. and 5% CO₂. At 24 h after transfection, the expression of transfected genes in cells was characterized by Western blot analysis. The cells were lysed in Mammalian Protein Extraction Reagent (M-PER) (Pierce, Rockford, Ill.) and the lysate protein concentrations were determined by spectrophotometry based on the manufacturer's instruction. Equal amounts (50 ug) of protein were loaded and separated by SDS-PAGE using a 10% polyacrylamide gel and blotted onto a nitrocellulose membrane. After blocking, the membrane was incubated with mouse anti-HPV-16 E6, E7 and rabbit anti-HPV 16 L2 polyclonal antibody for 1 hr at room temperature, washed and incubated with HRP-conjugated sheep anti-mouse Igs and donkey anti-rabbit Igs (Amersham Biosciences, UK), respectively. Membranes were detected with chemiluminescence ECL kit. (Amersham, Arlington Heights, Ill.).

### DNA Vaccination

For the gene gun-mediated intradermal vaccination, DNA-coated gold particles (1□g DNA/bullet) were delivered to the shaved abdominal region of C57BL/6 mice using a helium-driven gene gun (BioRad, Hercules, Calif., USA) with a discharge pressure of 400 p.s.i., according to a described protocol. C57BL/6 mice were vaccinated via a gene gun with 2 μg/mouse of pNGVL4a, pNGVL4a-hCRT, pNGVL4a-hCRTL2, pNGVL4a-hCRTE6E7 or pNGVL4a-hCRTE6E7L2. These mice received three boosters with the same dose and regimen at 1-week intervals. For the i.m.-mediated DNA vaccination, 50 □g/mouse of pNGVL4a, pNGVL4a-hCRT, pNGVL4a-hCRTL2, pNGVL4a-hCRTE6E7 and pNGVL4a-hCRTE6E7L2 DNA vaccines were delivered intramuscularly by syringe needle injection. These mice received three boosters with the same dose and regimen at 1-week intervals.

### Intracellular Cytokine Staining and Flow Cytometry Analysis

Cell surface marker staining of CD8 and intracellular cytokine staining for IFN-γ as well as FACScan analysis was performed using conditions described previously. Prior to FACScan, splenocytes from different vaccinated groups of mice were collected, pooled and incubated for 16 h with 1 ug/ml of E7 peptide (aa 49-57) or E6 peptide (aa 50-57) in the presence of GolgiPlug (1 ul/ml) for detecting E6 and E7-specific CD8⁺ T-cell precursors. The stimulated splenocytes were then washed twice with FACScan buffer. Cell surface marker staining for CD8 and intracellular cytokine staining for IFN-γ were performed using conditions described previously. The number of IFN-γ secreting CD8⁺ T cells was analyzed using flow cytometry. Analysis was performed on a Becton-Dickinson FACScan with CELLQuest software (Becton-Dickinson Immunocytometry System, Mountain View, Calif., USA).

### In Vivo Tumor Protection and Treatment Experiments Using TC-1 Tumors

For in vivo tumor protection experiments, C57BL/6 mice (5 per group) were vaccinated with 2 μg/mouse of pNGVL4a, pNGVL4a-hCRT, pNGVL4a-hCRTL2, pNGVL4a-hCRTE6E7 and pNGVL4a-hCRTE6E7L2 DNA vaccines by gene gun injection twice at a 1-week interval. One week after the last vaccination, mice were challenged with 5×10⁴ TC-1 tumor cells/mouse by subcutaneous injection in the right leg. Tumor growth was monitored by visual inspection and palpation twice a week as described. For in vivo tumor treatment experiments using an E6, E7-expressing tumor (TC-1), mice (5 per group) were intravenously challenged through the tail vein with 1×10⁴ TC-1 cells/mouse. At 3 days after tumor challenge, mice were administered 2 μg/mouse of pNGVL4a, pNGVL4a-hCRT, pNGVL4a-hCRTL2, pNGVL4a-hCRTE6E7 and pNGVL4a-hCRTE6E7L2 via gene gun. One week after the first vaccination, these mice were boosted with the same dose and regimen. Mice were killed and lungs were explanted on day 28. The pulmonary nodules on the surface of the lungs in each mouse were counted by experimenters blinded to sample identity as described.

### ELISA

The full-length L2 protein was expressed and purified as described. Briefly, the codon-modified full-length L2 was cloned into pET 28a vector and the His tagged fusion protein was expressed in BL21 (Rosetta cells. Novagen). The recombinant protein was purified on a Ni-NTA coumn under denaturing conditions following suggested manufacturer's protocol (Qiagen). The presence of anti-HPV-16 L2 antibodies in the sera was characterized by a direct ELISA as described. C57BL/6 mice were immunized with gene gun with 2 μg/mouse of the various DNA vaccines and received three boosters with the same dose and regimen at 1-week intervals. For intramuscular (i.m. or IM)-mediated DNA vaccination, 50 μg/mouse of each DNA vaccine was delivered intramuscularly by syringe needle injection. These mice received three boosters with the same dose and regimen at 1-week intervals. Sera were prepared from mice 7 days after last immunization and pooled. Full length of L2 protein (100 ng/well) was coated in a 96-microwell plate and incubated at 4° C. overnight. The wells were then blocked with PBS containing 1% BSA for 1 hour at 37° C. After washing with PBS containing 0.05% Tween-20, the plate was incubated with serially diluted sera (1:100, 1:500, 1:1000) for 2 hr at 37° C. Serum from vaccinated rabbit with full-length of L2 protein was used as the positive control. After washing twice with PBS containing 0.05% Tween-20, The plate was further mixed with 1:1,000 dilution of a HRP-conjugated donkey anti-rabbit IgG Ab for standard control and rabbit anti-mouse IgG Ab for mouse serum (Amersham Pharmacia Biotech, Piscataway, N.J., USA) as secondary antibody, respectively and was incubated at room temperature for 1 hour. The ELISA plate was read with a standard ELISA reader at 450 nm.

### Neutralization Assay

HPV16 pseudovirions with encapsulated secreted alkaline phosphatase (SEAP) were generated by co-transfection of 293TT cells with plasmids encoding HPV16 L2 and a SEAP reporter plasmid as described, e.g., by Buck et al. Cells collected after transfection were treated overnight with Brij 58 (0.5%), Benzonase (0.5%) and purified by centrifugation on an Optiprep step gradient (27, 33, and 39%) at 40,000 rpm for 4.5 h. Pseudovirus neutralization assays were carried out as outlined. Briefly, the pseudovirus and the pooled mouse immune sera were incubated for 1 h and the mixture was used to infect 293TT cells. 68 to 72 h post-infection, the supernatants were collected and SEAP activity in the supernatants was measured by colorimetric assay. Serum neutralization titers were defined as the highest dilution that caused at least a 50% reduction in SEAP activity, compared to control pre-immune serum samples. The minimum neutralization would be the wells where the virus is incubated with either pre-bled or PBS immunized serum and maximum neutralization would be the wells where the virus is completely neutralized and so there is no SEAP activity.

### Statistical Analysis

All data expressed as means ±s.d. are representative of at least two different experiments. Data for intracellular cytokine staining with flow cytometry analysis and tumor treatment experiments were analyzed by analysis of variance (ANOVA). Comparisons between individual data points were made using a Student's t-test. Kaplan Meier survival curves for tumor protection experiments were applied; for differences between curves, P-values were calculated using the log-rank test. The value of P<0.05 was considered significant.

### Results

Cells Transfected with the Various Recombinant DNA Constructs Express E6, E7 and/or L2 Proteins

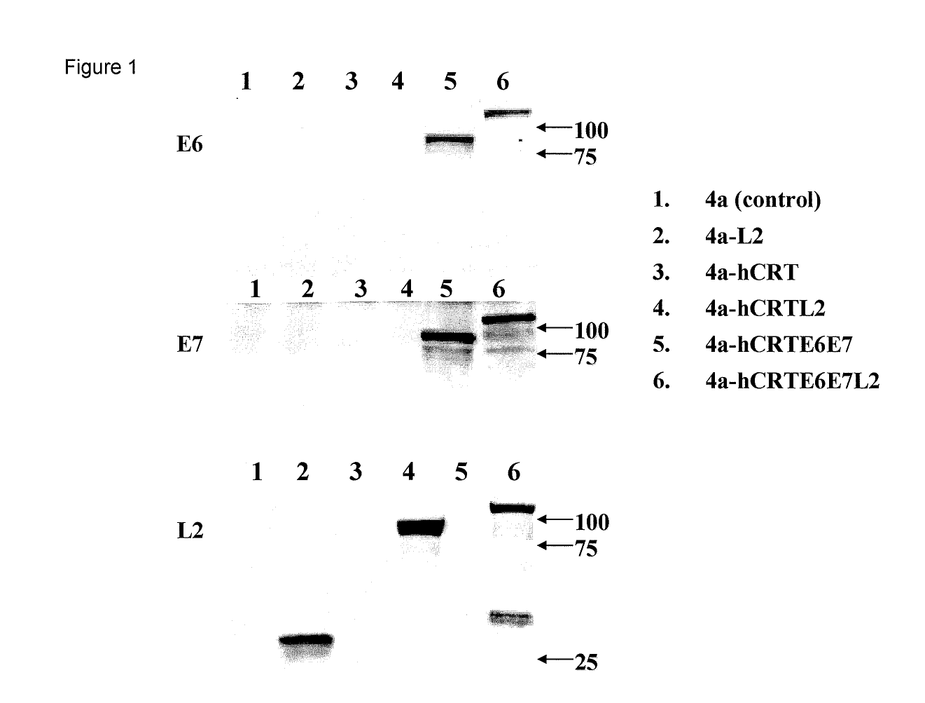

We generated the various DNA constructs pNGVL4a, pNGVL4a-L2, pNGVL4a-hCRT, pNGVL4a-hCRTL2, pNGVL4a-hCRTE6E7 and pNGVL4a-hCRTE6E7L2. In order to determine whether the BHK 21 cells transfected with each of recombinant DNA constructs expressed the encoded genes, E6, E7 and/or L2, we performed Western blot analysis with the cell lysates 24 hours after transfection using polyclonal antibodies to E6, E7 or L2 proteins. As shown in FIG. 1, expression of E6 (upper panel) was observed at comparable levels in the lysates of cells transfected with 4a-hCRTE6E7 (lane 5) and the 4a-hCRTE6E7L2 (lane 6) constructs. Similarly, the expression of E7 (middle panel) protein was observed at comparable levels in the lysates of cells transfected with 4a-hCRTE6E7 (lane 5) and the 4a-hCRTE6E7L2 (lane 6) constructs. Comparable level of expression of L2 protein (lower panel) was also observed in the lysates of cells transfected with 4a-L2 (lane 2), 4a-hCRTL2 (lane 4) and the 4a-hCRTE6E7L2 (lane 6) constructs. Furthermore, the proteins derived from each of the constructs were of the expected sizes. Thus, our data indicate that the BHK 21 cells transfected with the DNA constructs express the encoded genes for E6, E7 and/or L2.

Vaccination with DNA Vaccines Expressing hCRTE6E7 and hCRTE6E7L2 Generates Significant Number of E6 and E7-Specific CD8⁺ T Cells

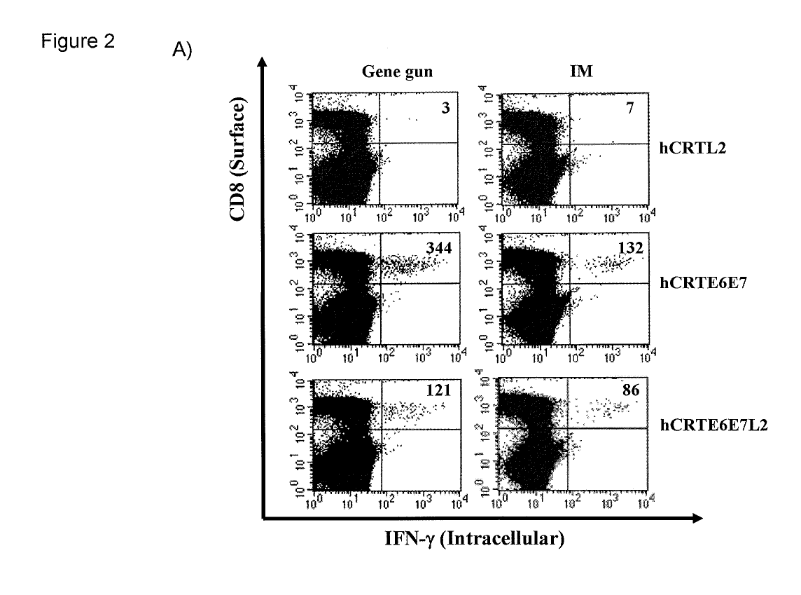

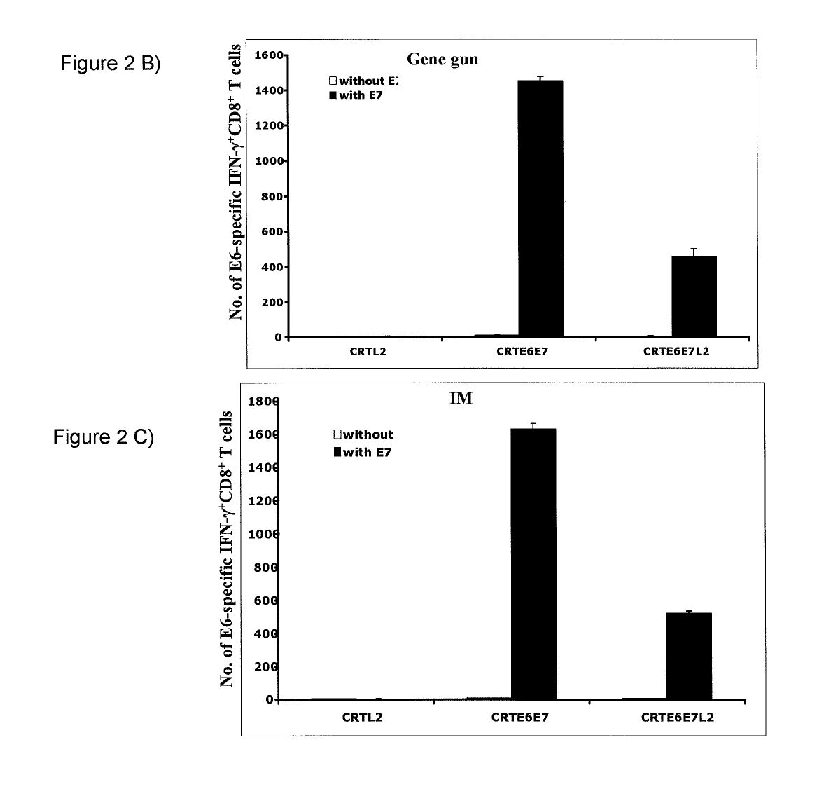

In order to assess the level of E6-specific T cell immune responses generated by the recombinant DNA vaccines, we first performed intracellular cytokine staining and flow cytometry analysis to determine the number of E6-specific IFN-γ secreting CD8+ T cells generated in response to stimulation with E6 peptide. C57BL/6 mice (3 per group) were immunized intradermally via gene gun (left panel) or intramuscularly (right panel) with the various DNA vaccines. The splenocytes from vaccinated mice were incubated with H-2^{K}-restricted E6 CTL peptide (aa 50-57) overnight and the number of E6-specific CD8+ T cells was determined. As shown in FIG. 2A, significant numbers of IFN-γ secreting CD8+ T cells were observed after stimulation with E6 peptide in the mice immunized with DNA vaccines expressing hCRTE6E7 and hCRTE6E7L2, although the immunization with DNA vaccines expressing hCRTE6E7L2 generates lower number of E6-specific IFN-γ+CD8+ T cells compared to immunization with DNA vaccines expressing hCRTE6E7. A graphical representation of the number of E6-specific IFN-γ+CD8+ T cells in mice immunized intradermally via gene gun and intramuscularly is depicted in FIGS. 2B and C respectively. In addition, this data indicate that immunization via gene gun generates a greater number of E6-specific IFN-γ+CD8+ T cells compared to intramuscular immunization. Thus, our data indicates that mice immunized with the DNA vaccines expressing hCRTE6E7 and hCRTE6E7L2 generate a higher frequency of E6-specific activated CD8+ T cells as compared to the hCRTL2 control.

We further characterized the level of E7-specific T cell immune responses generated by the recombinant DNA vaccines using intracellular cytokine staining and flow cytometry analysis. The splenocytes from vaccinated mice were incubated with H-2D^{b}-restricted E7 CTL peptide (aa 49-57) overnight and the number of E7-specific CD8+ T cells was determined. As shown in FIG. 3A, significant number of IFN-γ secreting CD8+ T cells were observed after stimulation with E7 peptide in the mice immunized with DNA vaccines expressing hCRTE6E7 and hCRTE6E7L2, although the immunization with DNA vaccines expressing hCRTE6E7L2 generates lower number of E7-specific IFN-γ+ CD8+ T cells compared to immunization with DNA vaccines expressing hCRTE6E7. A graphical representation of the number of E7-specific IFN-γ+ CD8+ T cells in mice immunized intradermally via gene gun and intramuscularly is depicted in FIGS. 3B and C respectively. In addition, this data indicate that immunization via gene gun and intramuscular immunization generate a similar number of E7-specific IFN-γ+ CD8+ T cells. Thus, our data indicates that the DNA vaccines expressing hCRTE6E7 and hCRTE6E7L2 generate a higher frequency of E7-specific activated CD8+ T cells as compared to the hCRTL2 control. Further, DNA vaccines expressing hCRTE6E7 and hCRTE6E7L2 generate detectable E6/E7-specific CD8+ T cell responses.

Vaccination with DNA Vaccines Expressing hCRTE6E7 and hCRTE6E7L2 Generates Significant Protection and Anti-Tumor Effect

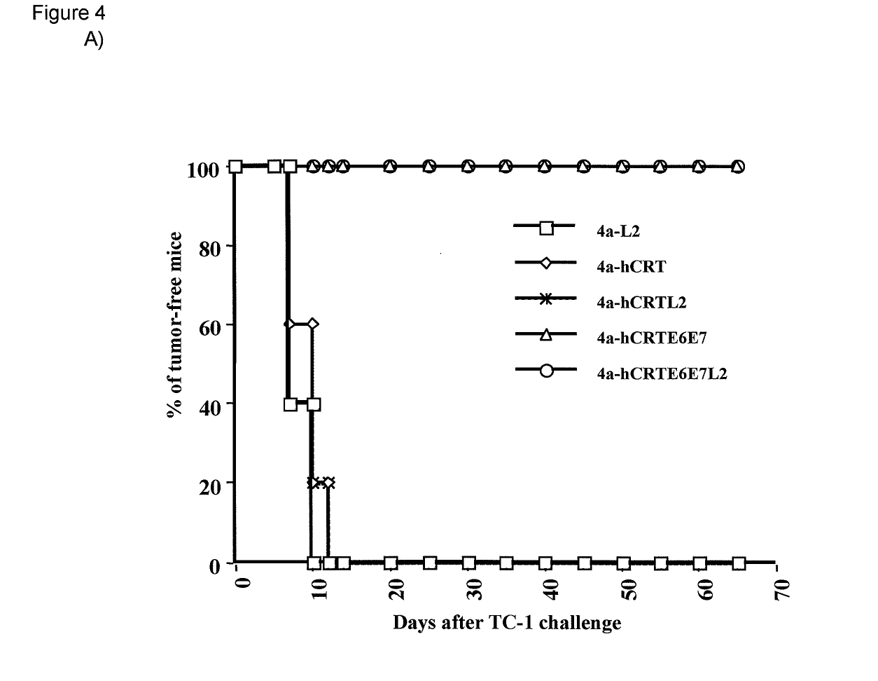

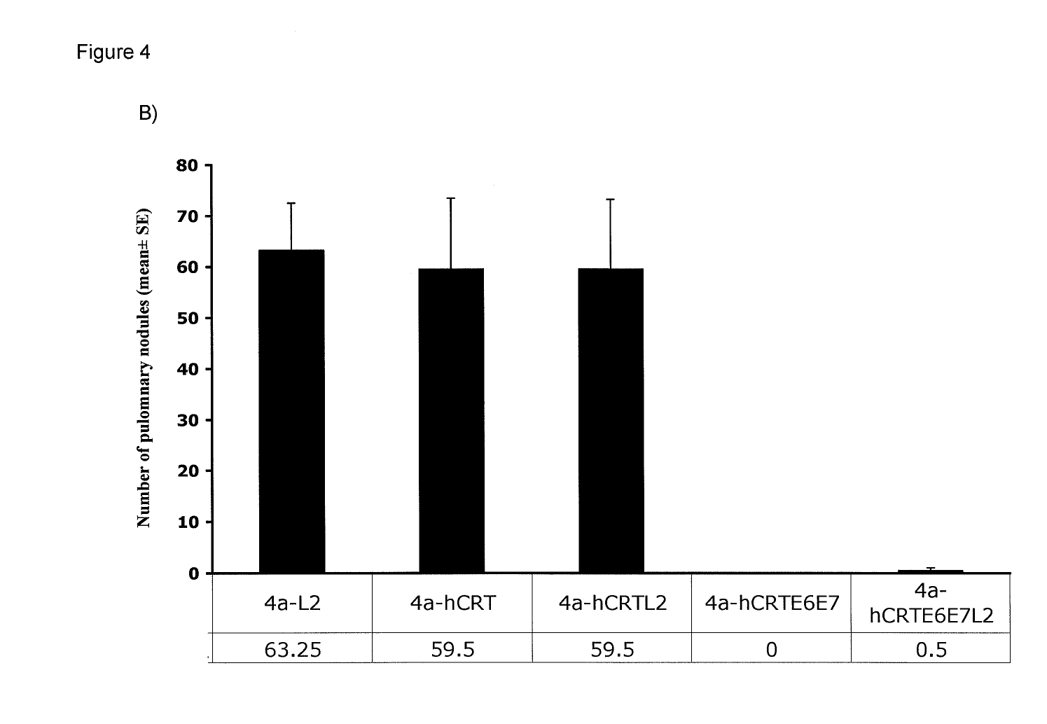

In order to assess the anti-tumor immunity generated by DNA vaccines expressing hCRTE6E7 and hCRTE6E7L2, we performed an in vivo tumor protection assay. C57BL/6 mice (5 per group) were immunized intradermally with the various DNA vaccines via gene gun twice at a one-week interval. Seven days after the last immunization, the mice were challenged subcutaneously with 5×10⁴ cells per mouse of TC-1 tumor cells. Tumor growth was monitored by visual inspection and palpation twice a week. As shown in FIG. 4A, immunization with DNA vaccines expressing hCRTE6E7 and hCRTE6E7L2 induced a higher percentage of tumor-free mice compared to immunization with the other DNA vaccines. In order to test the therapeutic effects of vaccination with DNA vaccines expressing hCRTE6E7 and hCRTE6E7L2, we challenged C57BL/6 mice (5 per group) intravenously with 1×10⁴ cells per mouse of TC-1 cells. Three and ten days later, the mice were treated intradermally with the various recombinant DNA vaccines via gene gun. Mice were sacrificed 28 days after tumor challenge and the numbers of pulmonary tumor nodules were quantified and compared. As shown in FIG. 4B, there was a significant reduction in the number of pulmonary tumor nodules of mice treated with DNA vaccines expressing hCRTE6E7 and hCRTE6E7L2 compared to the mice treated with the other DNA vaccines (p<0.01). Mice vaccinated with the DNA vaccines expressing hCRTE6E7 or hCRTE6E7L2 generate significant anti-tumor effects against E6/E7 expressing tumors.

Vaccination with DNA Vaccines Expressing hCRTE6E7L2 Generates Significant Levels of L2-Specific Neutralizing Antibody Responses

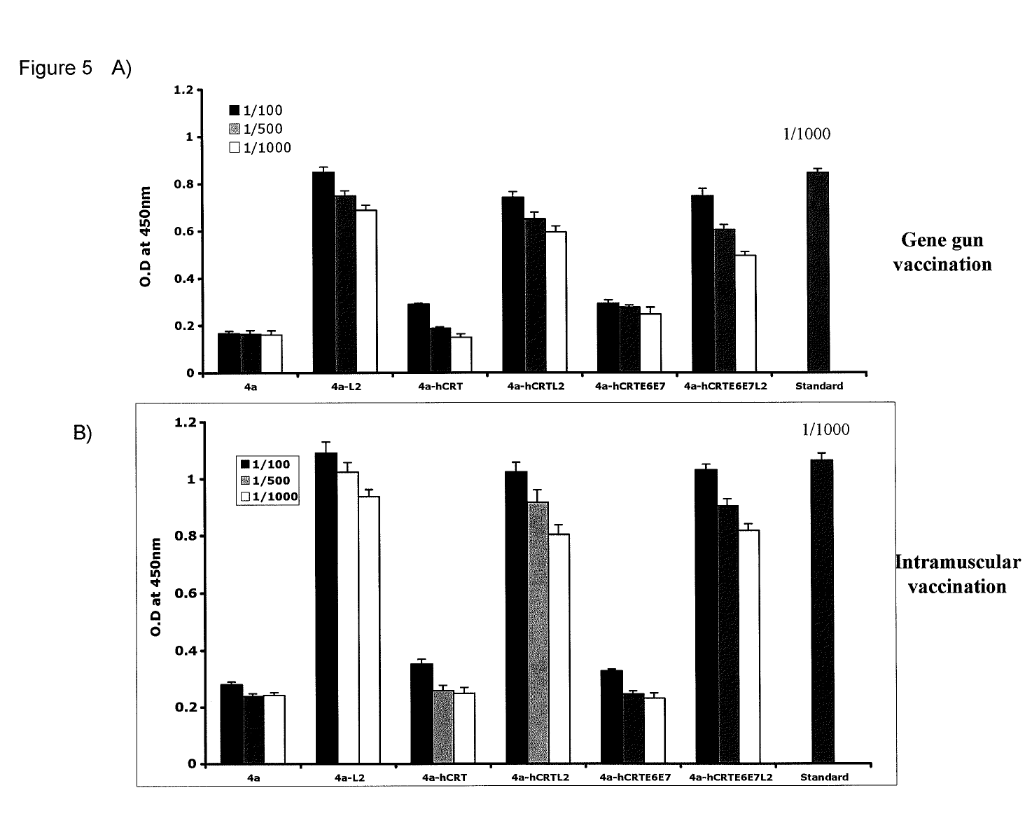

In order to determine the antibody levels against L2 obtained in the mice vaccinated with the various DNA vaccines, we performed ELISA using full length L2 protein. C57BL/6 mice (3 per group) were vaccinated intradermally via gene gun or intramuscularly with the various recombinant DNA vaccines. ELISA analysis was performed to determine the levels of L2-specific antibody in vaccinated mice. As shown in FIGS. 5A and B, there was a significant level of L2-specific antibody response in the mice vaccinated with DNA vaccine expressing hCRTE6E7L2 intradermally via gene gun or intramuscularly compared to that in mice vaccinated with DNA vaccine expressing hCRTE6E7. Furthermore, L2-specific antibody responses in mice vaccinated with the hCRTE6E7L2 DNA were comparable to the L2-specific antibody responses in mice vaccinated with 4a-L2 or 4a-hCRTL2 DNA (p>0.05). In addition, the L2-specific antibody responses in the mice vaccinated intramuscularly were comparable to that in mice vaccinated intradermally. Mice vaccinated with the DNA vaccine expressing hCRTE6E7L2 generate significant levels of L2-specific antibody.

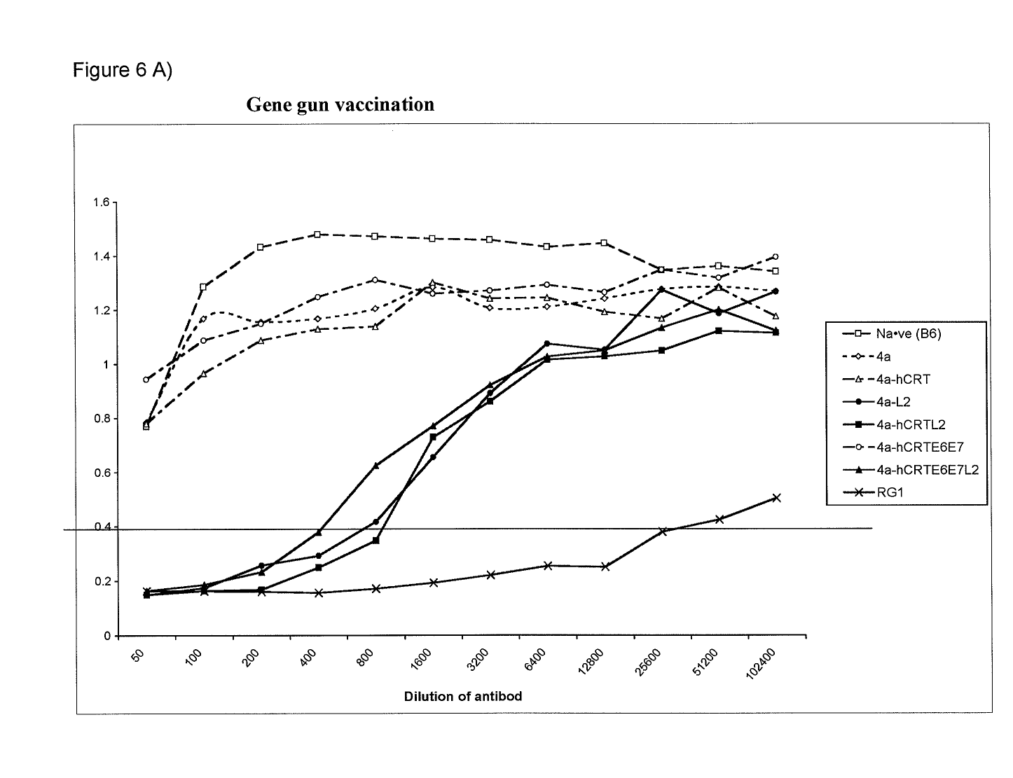

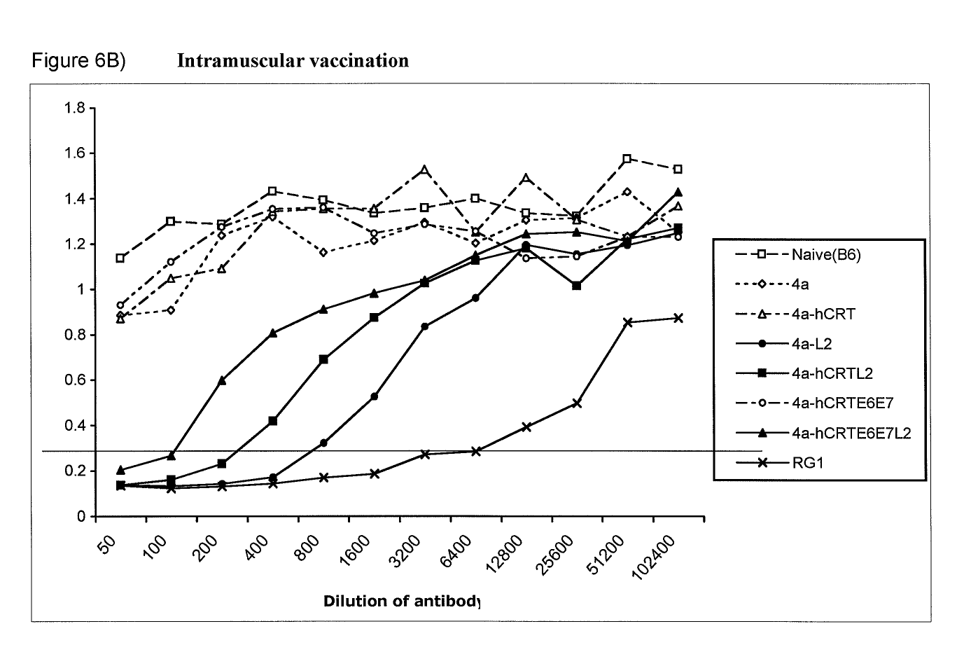

In order to determine the neutralizing antibody responses against HPV-16 pseudovirion infection in mice vaccinated with the various DNA vaccines, we performed neutralization assays using HPV-16 pseudovirions. C57BL/6 mice (3 per group) were vaccinated either intradermally via gene gun or intramuscularly with the various recombinant DNA vaccines. As shown in FIGS. 6A and B, there was a significant neutralizing antibody response in mice vaccinated with DNA vaccine expressing hCRTE6E7L2 compared to that in mice vaccinated with DNA vaccine expressing hCRTE6E7 (p<0.05). Furthermore, the neutralization activity observed in mice vaccinated with the hCRTE6E7L2 DNA was comparable to that in mice vaccinated with 4a-L2 DNA or 4a-hCRTL2 DNA. Intramuscular administration of the DNA vaccines showed that vaccination with hCRTE6E7L2 DNA generated a slightly lower titer of HPV-16 neutralizing antibodies compared to vaccination with 4a-L2 DNA vaccine. Therefore, hCRTE6E7L2 DNA is capable of generating significant levels of neutralizing antibody responses in vaccinated mice.

### Discussion

In the current study, we have created an HPV DNA vaccine encoding calreticulin linked to HPV16 early proteins, E6 and E7 and the late protein L2 (hCRTE6E7L2). Our data indicate that vaccination with hCRTE6E7L2 DNA vaccine is capable of generating a potent E6/E7-specific CD8+ T cell immune response and results in a significant therapeutic effect against E6/E7 expressing tumor cells. Furthermore, our data demonstrate that vaccination with hCRTE6E7L2 generated significant L2-specific neutralizing antibody responses against pseudovirion infection. Thus, we have created a preventive and therapeutic HPV-16 DNA vaccine that is an advancement of work using a HPV DNA vaccine containing pNGVL4a-hCRTE7, which uses a clinical grade gene gun device for patients with stage 1B1 HPV-16 positive cervical cancer. The present invention is an advancement of methods using hCRTE7 because in certain embodiments the vaccine targets both E6/E7 as well as L2. To address safety concerns regarding the potential for oncogenicity associated with administration of E6 and E7 as DNA vaccines into a subject such as a human, the invention provides embodiments in which attenuated (detox) versions of E6 and E7 are used that have been modified (e.g., by mutation). For example, a mutation at E7 position 24 and/or 26 will disrupt the Rb binding site of E7, abolishing the capacity of E7 to transform cells [32]. Furthermore, mutation at E6 positions 63 or 106 has been shown to destroy several HPV-16 E6 functions, preventing the mutated E6 protein from immortalizing human epithelial cells [33, 34]. Certain constructs used in the current invention employ attenuated (detox) forms of E6 and E7, thus eliminating any safety concerns that may arise.

Since the E6 and E7 proteins represent targets for the development of HPV therapeutic vaccines, DNA vaccines targeting both E6 and E7 generate a more potent immune response and anti-tumor effect than those targeting either E6 or E7 alone. However, the present invention provides DNA vaccines targeting either E6 or E7, or the combination of both E6 and E7 to generate a potent immune response and anti-tumor effect. A study by Peng et al. has demonstrated that simultaneous vaccination of C57BL/6 mice or HLA-A2 transgenic mice with both CRT/E6 and CRT/E7 DNA vaccines generates significant E6- and E7-specific T-cell immune responses in vaccinated mice. Furthermore, combined vaccination with both CRT/E6 and CRT/E7 DNA generates better therapeutic antitumor effects against HPV E6- and E7-expressing tumors than vaccination with either CRT/E6 DNA or CRT/E7 DNA alone [35].

The data described herein demonstrate that in certain embodiments, the gene gun-based immunization technique generates a higher number of antigen-specific CD8+ T cells compared to intramuscular immunization, particularly in case of E6 antigen. This is in agreement with studies by, e.g., Trimble et al. that indicate that vaccine administered via gene gun generated the highest number of antigen-specific CD8+ T cells of the methods tested. In addition, DNA vaccination via gene gun required the smallest dose to generate similar or slightly better antitumor effects compared to needle intramuscular and biojector administrations [36]. Thus, our data demonstrate that DNA vaccination via gene gun represents the an effective regimen for DNA administration.

Studies in the inventors' laboratories have shown that DNA vaccines encoding CRT linked to several antigens including HPV-16 E6, E7 or SARS-Co-V were capable of generating significantly higher levels of antigen-specific antibody responses compared to DNA vaccines encoding target antigen without linkage to CRT in vaccinated mice. In the current study, we observed that vaccination with DNA vaccine encoding CRT linked to L2 or E6E7L2 generated comparable levels of L2-specific antibody responses compared to vaccination with DNA vaccines encoding wild-type L2. While the CRT strategy did not enhance the L2-specific antibody responses, it did improve the antigen-specific CD8+ T cell-mediated immune response.

### REFERENCES

[1] Walboomers J M, Jacobs M V, Manos M M, Bosch F X, Kummer J A, Shah K V, et al. Human papillomavirus is a necessary cause of invasive cervical cancer worldwide. J Pathol 1999; 189(1):12-9.
[2] Ho G Y, Studentsov Y Y, Bierman R, Burk R D. Natural history of human papillomavirus type 16 virus-like particle antibodies in young women. Cancer Epidemiol Biomarkers Prev 2004; 13(1):110-6.
[3] Bosch F X, Lorincz A, Munoz N, Meijer C J, Shah K V. The causal relation between human papillomavirus and cervical cancer. J Clin Pathol 2002; 55(4):244-65.
[4] Ferenczy A, Franco E. Persistent human papillomavirus infection and cervical neoplasia. Lancet Oncol 2002; 3(1):11-6.
[5] zur Hausen H. Papillomaviruses and cancer: from basic studies to clinical application. Nature Rev Cancer 2002; 2(5):342-50.
[6] Roden R B, Yutzy WHt, Fallon R, Inglis S, Lowy D R, Schiller J T. Minor capsid protein of human genital papillomaviruses contains subdominant, cross-neutralizing epitopes. Virology 2000; 270(2):254-7.
[7] Gurunathan S, Klinman D M, Seder R A. DNA vaccines: immunology, application, and optimization. Annu Rev Immunol 2000; 18:927-74.
[8] Donnelly J J, Ulmer J B, Shiver J W, Liu M A. DNA vaccines. Annu Rev Immunol 1997; 15:617-48.
[9] Seder R A, Gurunathan S. DNA vaccines—designer vaccines for the 21st century. The New England journal of medicine 1999; 341(4):277-8.
[10] Nash P D, Opas M, Michalak M. Calreticulin: not just another calcium-binding protein. Molecular and cellular biochemistry 1994; 135(1):71-8.
[11] Basu S, Srivastava P K. Calreticulin, a peptide-binding chaperone of the endoplasmic reticulum, elicits tumor- and peptide-specific immunity. The Journal of experimental medicine 1999; 189(5):797-802.
[12] Conway E M, Liu L, Nowakowski B, Steiner-Mosonyi M, Ribeiro S P, Michalak M. Heat shock-sensitive expression of calreticulin. In vitro and in vivo up-regulation. The Journal of biological chemistry 1995; 270(28):17011-6.
[13] Spee P, Neefjes J. TAP-translocated peptides specifically bind proteins in the endoplasmic reticulum, including gp96, protein disulfide isomerase and calreticulin. Eur J Immunol 1997; 27(9):2441-9.
[14] Sadasivan B, Lehner P J, Ortmann B, Spies T, Cresswell P. Roles for calreticulin and a novel glycoprotein, tapasin, in the interaction of MHC class I molecules with TAP. Immunity 1996; 5(2): 103-14.
[15] Peng S, Ji H, Trimble C, He L, Tsai Y C, Yeatermeyer J, et al. Development of a DNA vaccine targeting human papillomavirus type 16 oncoprotein E6. J Virol 2004; 78(16):8468-76.
[16] Cheng W F, Hung C F, Chai C Y, Hsu K F, He L, Ling M, et al. Tumor-specific immunity and antiangiogenesis generated by a DNA vaccine encoding calreticulin linked to a tumor antigen. J Clin Invest 2001; 108(5):669-78.
[17] Kim T W, Lee J H, Hung C F, Peng S, Roden R, Wang M C, et al. Generation and characterization of DNA vaccines targeting the nucleocapsid protein of severe acute respiratory syndrome coronavirus. J Virol 2004; 78(9):4638-45.
[18] van der Burg S H, Kwappenberg K M, O'Neill T, Brandt R M, Melief C J, Hickling J K, et al. Pre-clinical safety and efficacy of TA-CIN, a recombinant HPV16 L2E6E7 fusion protein vaccine, in homologous and heterologous prime-boost regimens. Vaccine 2001; 19(27):3652-60.
[19] de Jong A, O'Neill T, Khan A Y, Kwappenberg K M, Chisholm S E, Whittle N R, et al. Enhancement of human papillomavirus (HPV) type 16 E6 and E7-specific T-cell immunity in healthy volunteers through vaccination with TA-CIN, an HPV16 L2E7E6 fusion protein vaccine. Vaccine 2002; 20(29-30):3456-64.
[20] Gambhira R, Gravitt P E, Bossis I, Stern P L, Viscidi R P, Roden R B. Vaccination of healthy volunteers with human papillomavirus type 16 L2E7E6 fusion protein induces serum antibody that neutralizes across papillomavirus species. Cancer Res 2006; 66(23): 11120-4.
[21] Leder C, Kleinschmidt J A, Wiethe C, Muller M. Enhancement of capsid gene expression: preparing the human papillomavirus type 16 major structural gene L1 for DNA vaccination purposes. J Virol 2001; 75(19):9201-9.
[22] Lin K Y, Guarnieri F G, Staveley-O'Carroll K F, Levitsky H I, August J T, Pardoll D M, et al. Treatment of established tumors with a novel vaccine that enhances major histocompatibility class II presentation of tumor antigen. Cancer Res 1996; 56(1):21-6.
[23] Chen C H, Wang T L, Hung C F, Yang Y, Young R A, Pardoll D M, et al. Enhancement of DNA vaccine potency by linkage of antigen gene to an HSP70 gene. Cancer Res 2000; 60(4):1035-42.
[24] Hung C F, Cheng W F, Hsu K F, Chai C Y, He L, Ling M, et al. Cancer immunotherapy using a DNA vaccine encoding the translocation domain of a bacterial toxin linked to a tumor antigen. Cancer Res 2001; 61(9):3698-703.
[25] Feltkamp M C, Smits H L, Vierboom M P, Minnaar R P, de Jongh B M, Drijfhout J W, et al. Vaccination with cytotoxic T lymphocyte epitope-containing peptide protects against a tumor induced by human papillomavirus type 16-transformed cells. Eur J Immunol 1993; 23(9):2242-9.
[26] Ji H, Wang T-L, Chen C-H, Hung C—F, Pai S, Lin K-Y, et al. Targeting HPV-16 E7 to the endosomal/lysosomal compartment enhances the antitumor immunity of DNA vaccines against murine HPV-16 E7-expressing tumors. Human Gene Therapy 1999; 10(17):2727-40.
[27] Gambhira R, Jagu S, Karanam B, Gravitt P E, Culp T D, Christensen N D, et al. Protection of Rabbits against Challenge with Rabbit Papillomaviruses by Immunization with the N Terminus of Human Papillomavirus Type 16 Minor Capsid Antigen L2. J Virol 2007; 81(21):11585-92.
[28] Cheng W F, Hung C H, Chai C Y, Hsu K F, He L, Ling M, et al. Enhancement of sindbis virus self-replicating RNA vaccine potency by linkage of herpes simplex virus type 1 VP22 protein to antigen. J Virol 2001; 75(5):2368-76.
[29] Buck C B, Pastrana D V, Lowy D R, Schiller J T. Efficient intracellular assembly of papillomaviral vectors. J Virol 2004; 78(2):751-7.
[30] Pastrana D V, Buck C B, Pang Y Y, Thompson C D, Castle P E, FitzGerald P C, et al. Reactivity of human sera in a sensitive, high-throughput pseudovirus-based papillomavirus neutralization assay for HPV16 and HPV18. Virology 2004; 321(2):205-16.
[31] Pastrana D V, Gambhira R, Buck C B, Pang Y Y, Thompson C D, Culp T D, et al. Cross-neutralization of cutaneous and mucosal Papillomavirus types with anti-sera to the amino terminus of L2. Virology 2005; 337(2):365-72.
[32] Munger K, Basile J R, Duensing S, Eichten A, Gonzalez S L, Grace M, et al. Biological activities and molecular targets of the human papillomavirus E7 oncoprotein. Oncogene 2001; 20(54):7888-98.
[33] Gao Q, Singh L, Kumar A, Srinivasan S, Wazer D E, Band V. Human papillomavirus type 16 E6-induced degradation of E6TP 1 correlates with its ability to immortalize human mammary epithelial cells. J Virol 2001; 75(9):4459-66.
[34] Dalal S, Gao Q, Androphy E J, Band V. Mutational analysis of human papillomavirus type 16 E6 demonstrates that p53 degradation is necessary for immortalization of mammary epithelial cells. J Virol 1996; 70(2):683-8.
[35] Peng S, Tomson T T, Trimble C, He L, Hung C F, Wu T C. A combination of DNA vaccines targeting human papillomavirus type 16 E6 and E7 generates potent antitumor effects. Gene Ther 2006; 13(3):257-65.
[36] Trimble C, Lin C T, Hung C F, Pai S, Juang J, He L, et al. Comparison of the CD8+ T cell responses and antitumor effects generated by DNA vaccine administered through gene gun, biojector, and syringe. Vaccine 2003; 21(25-26):4036-42.

The references cited above are all incorporated herein by reference, whether specifically incorporated or not. All publications, patents, patent applications, GenBank sequences and ATCC deposits, cited herein are hereby expressly incorporated by reference for all purposes. When definitions of terms in documents that are incorporated by reference herein conflict with those used herein, the definitions used herein govern. Citation of the documents herein is not intended as an admission that any of them is pertinent prior art. All statements as to the date or representation as to the contents of these documents is based on the information available to the applicant and does not constitute any admission as to the correctness of the dates or contents of these documents. Having now fully described this invention, it will be appreciated by those skilled in the art that the same can be performed within a wide range of equivalent parameters, concentrations, and conditions without departing from the spirit and scope of the invention and without undue experimentation.

1. A nucleic acid composition, comprising:
(a) a first nucleic acid comprising a first sequence encoding an E6 or E7 protein of a human papillomavirus (HPV), wherein linked to the first sequence, directly or via a linker, is a second sequence that encodes an HPV late protein L2; and
(b) a second nucleic acid encoding a calreticulin,
wherein the first nucleic acid and the second nucleic acid are operably linked either directly or via a linker.

2. A nucleic acid composition, comprising:
(a) a first sequence encoding a human papillomavirus (HPV) E6 protein or an immunogenically active fragment thereof;
(b) a second sequence encoding a HPV E7 protein or an immunogenically active fragment thereof;
(c) a third sequence encoding an HPV late protein L2 or an immunogenically active fragment thereof; and
(d) a fourth sequence encoding a calreticulin or an immunogenically active fragment thereof.

3. The composition of claim 2, wherein the HPV is HPV-16, and wherein the calreticulin is human calreticulin.

4. A DNA vaccine composition comprising a plasmid vector comprising the nucleic acid composition of claim 2 and an immunologically acceptable excipient or carrier.

5. A particle suitable for introduction into a cell or an animal, to which particle is bound the nucleic acid composition of claim 2.

6. The particle of claim 5, comprising a gold particle.

7. A method of inducing or enhancing an antigen-specific immune response in a mammalian subject, comprising administering to the subject an effective amount of the composition of claim 2, thereby inducing or enhancing the antigen specific immune response.

8. A method of inducing or enhancing an antigen-specific immune response in a mammalian subject, comprising administering to the subject an effective amount of the particle of claim 5, thereby inducing or enhancing the antigen specific immune response.

9. The method of claim 8, wherein the antigen specific immune response is mediated at least in part by CD8⁺ cytotoxic T lymphocytes (CTL).

10. The method of claim 8, wherein the antigen specific immune response is mediated at least in part by CD8⁻ cytotoxic T lymphocytes (CTL).

11. The method of claim 8, wherein the mammalian subject is a human.

12. The method of claim 8, wherein the particle is administered intradermally by particle bombardment.

13. The method of claim 8, wherein the mammalian subject is a human having a tumor, and wherein the particle is administered intratumorally or peritumorally.

14. The method of claim 8, wherein the immune response is (i) specific for HPV E6 or E7 protein or immunogenically active fragment thereof, and (ii) greater in magnitude than an immune response induced by a DNA that encodes HPV E6, E7 and L2 without a DNA encoding the calreticulin or fragment thereof.

15. The composition of claim 2, wherein the first sequence encodes a HPV E6 protein comprising an amino acid sequence selected from the group consisting of

| | |
|---|---|
| LSRHFMHQKRTAMFQDPQERPRKILPQ | (SEQ ID NO: 13) |
| and | |
| AMFQDPQERPRKLPQLCTELQTTIHDIILEC. | (SEQ ID NO: 14) |

16. The composition of claim 2, wherein the second sequence encodes a HPV E7 protein comprising an amino acid sequence selected from the group consisting of

| | |
|---|---|
| PTLHEYMLDLQPETTDLYCYEQ, | (SEQ ID NO: 15) |
| HEYMLDLQPET | (SEQ ID NO: 16) |
| TLHEYMLDLQPETTD, | (SEQ ID NO: 17) |
| EYMLDLQPETTDLY, | (SEQ ID NO: 18) |
| DEIDGPAGQAEPDRAHY | (SEQ ID NO: 19) |
| and | |
| GPAGQAEPDRAHYNI. | (SEQ ID NO: 20) |

17. The composition of claim 2, wherein the third sequence encodes a HPV L2 protein comprising an amino acid sequence selected from the group consisting of
(SEQ ID NO: 21)
TGVPIDPAVPDSSIVPLLES,
(SEQ ID NO: 22)
GAEIEIAEVHPPPVYEGPE,
(SEQ ID NO: 23)
VTIGDIEEPPILEVVPETHPT
and
(SEQ ID NO: 24)
SRMKRASATQLYKTCKQAGTCPPDIISKVEGKTIADQILQYGSMGVFFGG
LGIGTGSGTGGRTGYIPLGTRPPTATDTLA.

18. The composition of claim 2, wherein the fourth sequence encodes a calreticulin protein comprising the amino acid sequence
(SEQ ID NO: 26)
MLLSVPLLLGLLGLAVAEPAVYFKEQFLDGDGWTSRWIESKHKSDFGKFV
LSSGKFYGDE.

19. A nucleic acid composition comprising SEQ ID NO: 1.

20. A nucleic acid composition encoding an amino acid sequence comprising SEQ ID NO: 2.
